SEQUENCE LISTING (17 sequences)

SEQ ID NO: 1 - protein, 528 aa, Artificial Sequence
  Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 2 - protein, 533 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDAAHLAASPPPLALLAGAMLLLLAPTLY

SEQ ID NO: 3 - protein, 530 aa, Artificial Sequence
  Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLPAPSGLSDAAHLAASPPSLALLAGAMLLLLAPALY

SEQ ID NO: 4 - protein, 503 aa, Artificial Sequence
  Synthetic polymer.
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPAGTTD

SEQ ID NO: 5 - protein, 506 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 6 - protein, 503 aa, Artificial Sequence
  Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLPAPSGLSD

SEQ ID NO: 7 - protein, 508 aa, Artificial Sequence
  Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLPAPSGLSDAAHLA

SEQ ID NO: 8 - protein, 754 aa, Artificial Sequence
  Synthetic polymer.
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 751 aa, Artificial Sequence
  Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLPAPSGLSDGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 539 aa, Artificial Sequence
  Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLEVLFQGPAPPAGTTDAAHPGRSVVPALLPLRAGTLLLLETATAP

SEQ ID NO: 11 - protein, 534 aa, Artificial Sequence
  Synthetic polymer.
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLAPPAGTTDAAHPIEGRSVVPALLPLRAGTLLLLETATAP

SEQ ID NO: 12 - DNA, 1669 bp, Artificial Sequence
  Synthetic polymer.
atgcaggggccctgggtgctgctgctgctgggcctgaggctacagctctccctgggcgtcatcccaggtaatgaggctccccaagctgttccacacacagggcaccccctcagccaggctgacctgatctctactctccccctggccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctga

SEQ ID NO: 13 - DNA, 1671 bp, Artificial Sequence
  Synthetic polymer.
atgcaggggccctgggtgctgctgctgctgggcctgaggctacagctctccctgggcgtcatcccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctgatttactaaaaccttgaaataaaattgtaaaacatcagtttgaaggcctgactctcagggtagttcttttttaattctgggtttt

SEQ ID NO: 14 - DNA, 1672 bp, Artificial Sequence
  Synthetic polymer.
atgcagtgggcctgtgtgctgctgctgctgggcctgtggctacagctctccctcaccttcatcccaggtaatcaggcggctcccagcagcccctactcacaggggcggctctaggctgacctgaccaacactctccccttgggcagctgaggaggaagaccccgccttctggaaccgccaggcagcccaggcccttgatgtagccaagaagttgcagccgatccagacagctgccaagaatgtcatcctcttcttgggggatgggatgggggtgcctacggtgacagccactcggatcctaaaggggcagatgaatggtaagctgggacctgagacacccctggccatggaccagttcccatacgtggctctgtccaagacatacaacgtggacagacaggtgccagacagcgcaggcactgccactgcctacctgtgtggggtcaagggcaactacaaaaccattggtgtaagtgcagccgcccgctacaaccagtgcaacacaacaagtggcaatgaggtcacgtctgtgatgaaccgggccaagaaagcaggaaagtcagtgggagtggtgaccacctccagggtgcagcatgcctccccagccggtgcttatgcacacacggtgaaccgaaactggtactcagatgccgacctgcctgccgatgcacagacgtatggctgccaggacatcgccacacaactggtcaacaacatggatattgacgtgatcctgggtggaggccgaatgtacatgtttcctgaggggaccccggatcctgaatacccatacgatgtcaatcagactggagtccggaaggacaagcggaatctggtgcaggagtggcaggccaagcaccagggagcccagtatgtgtggaaccgcacggagctccttcaggcagccaatgaccccagtgtaacacacctcatgggcctctttgagccggcagacatgaagtataatgttcagcaagaccccaccaaggacccgaccctggaggagatgacggaggcggccctgcaagtgctgagcaggaacccccagggcttctacctcttcgtggagggaggccgcattgaccacggtcaccatgaaggcaaagcttatatggcactgactgatacagtcatgtttgacaatgccatcgccaaggctaacgagctcactagcgaactggacacgctgatccttgccactgcagaccactcccatgtcttctcttttggtggctacacactgcgtgggacctccattttcggtctggcccccagcaaggcctcagacaacaagtcctacacctccatcctctatggcaatggccctggctacgtgcttggtgggggcttaaggcccgatgttaatgacagcataagcgaggacccctcgtaccggcagcaggcggccgtgcccctgtctagtgagtcccacgggggcgaggacgtggcggtgttcgcgcgaggcccgcaggcgcacctggtgcacggcgtgcaggaggagaccttcgtggcgcacgtcatggcctttgcgggctgcgtggagccctacaccgactgcaatctgccggccccctctggcctctccgacgccgcgcacctggcggccagcccgccttcgctggcgctgctggccggggcgatgctgctgctgctggcgcctgccttgtactga

SEQ ID NO: 15 - DNA, 1869 bp, Artificial Sequence
  Synthetic polymer.
atgcagtgggcctgtgtgctgctgctgctgggcctgtggctacagctctccctcaccttcatcccagctgaggaggaagaccccgccttctggaaccgccaggcagcccaggcccttgatgtagccaagaagttgcagccgatccagacagctgccaagaatgtcatcctcttcttgggggatgggatgggggtgcctacggtgacagccactcggatcctaaaggggcagatgaatggtaagctgggacctgagacacccctggccatggaccagttcccatacgtggctctgtccaagacatacaacgtggacagacaggtgccagacagcgcaggcactgccactgcctacctgtgtggggtcaagggcaactacaaaaccattggtgtaagtgcagccgcccgctacaaccagtgcaacacaacaagtggcaatgaggtcacgtctgtgatgaaccgggccaagaaagcaggaaagtcagtgggagtggtgaccacctccagggtgcagcatgcctccccagccggtgcttatgcacacacggtgaaccgaaactggtactcagatgccgacctgcctgccgatgcacagacgtatggctgccaggacatcgccacacaactggtcaacaacatggatattgacgtgatcctgggtggaggccgaatgtacatgtttcctgaggggaccccggatcctgaatacccatacgatgtcaatcagactggagtccggaaggacaagcggaatctggtgcaggagtggcaggccaagcaccagggagcccagtatgtgtggaaccgcacggagctccttcaggcagccaatgaccccagtgtaacacacctcatgggcctctttgagccggcagacatgaagtataatgttcagcaagaccccaccaaggacccgaccctggaggagatgacggaggcggccctgcaagtgctgagcaggaacccccagggcttctacctcttcgtggagggaggccgcattgaccacggtcaccatgaaggcaaagcttatatggcactgactgatacagtcatgtttgacaatgccatcgccaaggctaacgagctcactagcgaactggacacgctgatccttgccactgcagaccactcccatgtcttctcttttggtggctacacactgcgtgggacctccattttcggtctggcccccagcaaggcctcagacaacaagtcctacacctccatcctctatggcaatggccctggctacgtgcttggtgggggcttaaggcccgatgttaatgacagcataagcgaggacccctcgtaccggcagcaggcggccgtgcccctgtctagtgagtcccacgggggcgaggacgtggcggtgttcgcgcgaggcccgcaggcgcacctggtgcacggcgtgcaggaggagaccttcgtggcgcacgtcatggcctttgcgggctgcgtggagccctacaccgactgcaatctgccggccccctctggcctctccgacgccgcgcacctggcggccagcccgccttcgctggcgctgctggccggggcgatgctgctgctgctggcgcctgccttgtactgaggggacccgggggtggggacacaggccccgccctccctgggaggcaggaagcagctctcaaataaactgttctaagtatgatacaggagtgatacatgtgtgaagagaagcccttaggtgggggcacagagtgtctgggtgaggggggtcagggtcacatcaggaggttagggaggggttgatgaagggctgacgttgagcaaagaccaaaggcaactcagaaggacagtggtgcaggactgggtgtggtcagcagggggactggttgggggatcc

SEQ ID NO: 16 - DNA, 1259 bp, Artificial Sequence
  Synthetic polymer.
aaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgaacaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcaggaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaa

SEQ ID NO: 17 - protein, 420 aa, Artificial Sequence
  Synthetic polymer.
KKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKNRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

(19) United States
(12) Patent Application Publication
(10) Pub. No.: US 2021/0189358 A1
(43) Pub. Date: Jun. 24, 2021

(54) METHODS OF MAKING ALKALINE PHOSPHATASE AGENTS

(71) Applicant: Synthetic Biologics, Inc., Rockville, MD (US)

(72) Inventors: Maria RODRIGUES, Monthey (CH); Veronique DE VAUX, Monthey (CH); Michael KALEKO, Rockville, MD (US)

(21) Appl. No.: 17/252,447

(22) PCT Filed: Jun. 17, 2019
(86) PCT No.: PCT/US19/37419

§ 371 Date: Dec. 15, 2020

Related U.S. Application Data
(60) Provisional application No. 62/686,467, filed on Jun. 18, 2018.

(51) Int. Cl. C12N 9/16; C12N 5/071

(52) U.S. Cl. CPC C12N 9/16; C12N 5/0682; C12Y 301/03001; C12N 2523/00; C12N 2500/22; C12N 2500/32; C12N 2500/50; C12N 2500/16

(57) ABSTRACT

The present invention relates to, inter alia, compositions and methods, including therapeutic alkaline phosphatases that find use in the treatment of disease, such as microbiome-related diseases. In part, the invention provides, in various embodiments, methods of manufacturing therapeutic alkaline phosphatases.

### CROSS-REFERENCE TO RELATED APPLICATIONS

This application claims the benefit of U.S. Provisional Application No. 62/686,467, filed Jun. 18, 2018, the entire contents of which are hereby incorporated by reference in their entirety.

### FIELD OF THE INVENTION

The present invention provides, in part, improved methods of manufacturing of therapeutic intestinal alkaline phosphatases that find use in the treatment of disease, such as microbiome-related diseases. Throughout this application, the terms microbiome and microbiota are used interchangeably.

### DESCRIPTION OF THE TEXT FILE SUBMITTED ELECTRONICALLY

The content of the text file submitted electronically herewith is incorporated herein by reference in their entirety: A computer readable format copy of the Sequence Listing (Filename: “SYN-037PC_ST25.txt”; Date created: Jun. 12, 2019; File size: 67.5 KB).

### BACKGROUND

Alkaline phosphatases (“APs,” EC 3.1.3.1) are dimeric metalloenzymes that catalyze the hydrolysis of phosphate esters and dephosphorylate a variety of target substrates optimally at physiological and higher pHs. Alkaline phosphatases (APs) are found in prokaryotic as well as in eukaryotic organisms (e.g., in E. coli and mammals). Mammalian APs have been shown to play important roles in gut hemostasis, mucosal barrier function, promotion of commensal bacteria, and defense from pathogens. Mammalian APs exert their properties by primarily targeting lipopolysaccharide (LPS, a toll-like receptor-4 (TLR4) agonist), flagellin (a TLRS agonist) and CpG DNA (a TLR9 agonist). APs also degrade intestine luminal nucleotide triphosphates (NTPs, e.g., ATP, GTP, etc.), which promotes the growth of good bacteria and reverses dysbiosis. Accordingly, APs may find clinical use as, for example, microbiome preserving agents for treating various gastrointestinal (GI) disorders. Because intestinal alkaline phosphatase IAP is a naturally occurring gut enzyme, it can be safely administered orally and does not appear to be associated with side effects (Alam, S N, Yammine, H, Moaven, O, Ahmed, R., Moss, A K, Biswas, B, Muhammad, N, Biswas, R, Raychowdhury, A, Kaliannan, K, Ghosh, S, Ray, M, Hamarneh, S, Barua, S, Malo, N S, Bhan, A K, Malo, M S Hodin, R A. (2014); Lukas, M, Drastich, Pavel, Konecny, M, Gionchetti, P, Urban, O, Cantoni, F, Bortlik, M, Duricova, D, Bulitta M, Inflamm Bowel Dis, 16:1180-1186 (2010)). Intestinal alkaline phosphatase prevents antibiotic-induced susceptibility to enteric pathogens. Ann Surg. 259:715-722.

However, AP has been difficult to express to high levels in various cellular production systems. Mainly groups have focused on expressing placental alkaline phosphatase (PLAP) or tissue nonspecific alkaline phosphatase (TNAP). Expression has been tested using mammalian cells (COS; Berger, J, Howard, A D, Gerger, L, Cullen, B R, Udenfriend, S. (1987). Expression of active, membrane-bound human placental alkaline phosphatase by transfected simian cells. Proc. Natl. Acad. Sci USA 84:4885-4889; and CHO, Millan, J L. Mammalian alkaline Phosphatases. Wiley-VCH Verlag GmbH & Co. 2006), insect cells (baculovirus system, TNAP, Oda, K, Amaya, Y, Fukushi-Irie, M, Kinameri, Y, Ohsuye, K, Kubota, I, Fujimura, S, Kobayashi, J. (1999). A general method for rapid purification of soluble versions of glycosylphosphatidylinositol-anchored proteins expressed in insect cells: an application for human tissue-nonspecific alkaline phosphatase. J. Biochem. 126:694-699; PLAP Zhang, F., Murhammer, D S, Linhardt, R J. (2002). Enzyme kinetics and glycan structural characterization of secreted alkaline phosphatase prepared using the baculovirus expression vector system. Appl. Biochem. Biotechnol. 101:197-210, expression of PLAP was 7 U/mL in Tn-5B1 cells and 4.1 U/mL in Sf9 cells), yeast (Pichia pastoris, PLAP, 2 mg/L, Heimo, H K, Palmu, K, Suominen, I. (1998). Human placental alkaline phosphatase: expression in Pichia pastoris, purification and characterization of the enzyme. Protein Expr. Purif. 12:85-92), bacteria (PLAP, Beck, R, Burtscher, H. (1994). Expression of human placental alkaline phosphatase in Escherichia coli. Protein Expr. Purif. 5:192-197, most protein was insoluble and was 5% of the total protein in the cell), transgenic tobacco (PLAP, Komarnytsky, S., Borisjuk, N V, Borisjuk, L G., Alam, M Z, Raskin, I. (2000). Production of recombinant proteins in tobacco guttation fluid. Plant Physiol. 124:927-933, transgenic tobacco, up to 1.1 ug/g dry leaf weight/day secreted into the guttation fluid), and tobacco NT1 cell suspension cultures (PLAP, Becerra-Arteaga, A, Mason, H S Shuler, M L. (2006). Production, secretion, and stability of human secreted alkaline phosphatase in tobacco NT1 cell suspension cultures. Biotechnol. Prog. 22:1643-1649; up to 0.4 U/mL or 27 mg/L). Fewer groups focused on expressing IAP but similar to PLAP and TNAP expression levels, IAP expression was extremely low for recombinant expression systems. For example, expression of bovine IAP II in the yeast, Pichia pastoris, (Roche-Bretthauer and Castellino. Biotechnol Appl Biochem, 1999. Glycosylation of Pichia pastoris-derived proteins, 3:193-200) and in the protist, Tetrahymena thermophilia (hIAP, 14,000 U/L in 2 days, Aldag, I, Bockau, U, Rossdorf, J, Laarmann, S, Raaben, W, Hermann, L, Weide, T, Hartman MWW. (2011). Expression, secretion and surface display of a human alkaline phosphatase by the ciliate Tetrahymena thermophilia. BMC Biotechnology 11:11) was low. Other reports of expression of IAP in Chinese hamster ovary (CHO) cells, either stably transformed or transiently transfected did not report expression levels, but they are assumed to be low (Millan, J L. Mammalian alkaline Phosphatases. Wiley-VCH Verlag GmbH & Co. 2006). In a human amniocyte cell line, transient recombinant protein was expressed up to 0.5 g/L (Fischer, S, Charara, N, Gerber, A, Wolfel, J, Schiedner, G, Voedisch, B, Geisse, S, Biotech and Bioeng, 109:2250-2261 (2012),

Therefore, there remains a need for methods to manufacture sufficient AP drug product for clinical use.

### SUMMARY OF THE INVENTION

Accordingly, in some embodiments, the present invention provides various recombinant AP constructs (“AP-based agents”) and therapeutic uses thereof in which the AP constructs are manufactured using protein expression systems, such as expression in cell lines, including mammalian cell lines, in bioreactors that provide commercially adequate AP activity yields. Surprisingly, it was found that addition of supplementary zinc carriers (e.g., ZnSO₄, ZnCl₂, hydrolysates, or serum albumins) during the culturing process produced AP-based agents with higher total AP activity and specific activity, as compared to AP-based agents produced without such a step.

In one aspect, the present invention provides for the production of an AP-based agent in cell lines, such as mammalian cells. The method includes providing a host cell transformed with a vector comprising a sequence encoding the AP-based agent. The cell is grown in a bioreactor to induce expression of the AP-based agent. In an embodiment, the host cell is CHO cell. In various embodiments, methods of the invention allow for production of the AP-based agent having high total AP activity and high specific activity.

In various embodiments, the AP-based agent is a mammalian or bacterial alkaline phosphatase. In some embodiments, the AP-based agent is a mammalian alkaline phosphatase. In an embodiment, the AP-based agent is an intestinal alkaline phosphatase. In some embodiments, the AP-based agent is a bacterial alkaline phosphatase. In some embodiments, the bacterial alkaline phosphatase has catalytic activity comparable to that of a mammalian phosphatase. In some embodiments, the AP-based agent is secreted from the host cell.

In another aspect, the present invention provides methods for the therapeutic use of the AP-based agent produced as described herein. In an embodiment, the present invention provides methods for the treatment of a microbiome-related disorder. In another embodiment, the present invention provides methods for the treatment or prevention of an antibiotic-induced adverse effect in the GI tract and/or a C. difficile infection (CDI) and/or a C. difficile-associated disease. In another embodiment, the present invention provides methods for the treatment of a metabolic disorder such as obesity, diabetes, and/or a metabolic syndrome. In another embodiment, the present invention provides methods for the treatment of a neurological disease and neuropsychiatric disorders (i.e. autism spectrum disorders, anxiety-related disorders). Methods for treating sepsis and acute kidney injury (AM) and renal failure are also provided. In a further embodiment, the present invention provides methods for the treatment of HIV-mediated gut dysbiosis and/or GI barrier dysfunction. In another embodiment, the present invention provides methods for the prevention or treatment of autoimmune disorders and IBD, for example, Celiac disease, Crohn's disease, acute radiation enteropathy, chronic delayed radiation enteropathy, proctitis, and colitis (e.g., ulcerative colitis). In another embodiment, the present invention provides methods for preventing, treating as well as working as adjuvant in cancer immunotherapy applications.

### DESCRIPTION OF THE FIGURES

FIG. 1 depicts sequences pertaining to alkaline phosphatase agents present in the manufacturing processes described herein.

FIG. 2 depicts results from various bioreactor process runs under Condition 1, 2, or 3, on day 14 of the culturing process. Measurements were taken of viable cell density (VCD) in cells/mL, integrated viable cell density (IVCD) in cells/mL*day, BIAP titer in g/L, percent dimers, final volume in Liters, total BIAP in grams, AP activity in U/mL, specific activity in U/mg, total active AP units, and average cell productivity in pg/cell/day. The results show that the IAP produced from Bioreactor 7 (BR7, under Condition 3) exhibited higher AP activity, specific activity, and total active AP units on Day 14, as compared to the IAP produced from bioreactors under Conditions 1 and 2.

FIG. 3 depicts AP activity (U/mL) results from various bioreactor process runs under Condition 1, 2, or 3, on days 12, 13, and 14 of the culturing process. In each series of histograms, the left bar is day 12, the middle bar is day 13, and the right bar is day 14. The results show that the IAP produced from Bioreactor 7 (Seeding 5×10⁵+Zn, under Condition 3) exhibited higher AP activity on days 12, 13, and 14, as compared to the IAP produced from Conditions 1 and 2.

FIG. 4 depicts specific activity (U/mg) results from various bioreactor process runs under Condition 1, 2, or 3, on days 12, 13, and 14 of the culturing process. In each series of histograms, the left bar is day 12, the middle bar is day 13, and the right bar is day 14. The results show that the IAP produced from Bioreactor 7 (Seeding 5×10⁵+Zn, under Condition 3) exhibited higher specific activity on days 12, 13, and 14, as compared to the IAP produced from Conditions 1 and 2.

FIG. 5 depicts total active AP units from various bioreactor process runs under Condition 1, 2, or 3, on days 12, 13, and 14 of the culturing process. In each series of histograms, the left bar is day 12, the middle bar is day 13, and the right bar is day 14. The results show that the IAP produced from Bioreactor 7 (Seeding 5×10⁵+Zn, under Condition 3) exhibited higher total active AP units, as compared to the IAP produced from Conditions 1 and 2.

FIG. 6 depicts depicts metabolite content, pH, and osmolality on day 14 for IAP produced under all three bioreactor culturing conditions. Measured metabolites include glutamine, glucose, lactate, ammonium, sodium, and potassium.

FIG. 7 depicts pH measurements over the course of the bioreactor process for IAP produced under all three conditions.

FIG. 8 depicts osmolality as measured over the course of the bioreactor process for IAP produced under all three conditions.

FIG. 9 depicts glucose levels as measured over the course of the bioreactor process for IAP produced under all three conditions.

FIG. 10 depicts ammonium levels (mM) as measured over the course of the bioreactor process for IAP produced under all three conditions.

FIG. 11 depicts lactate levels (g/L) as measured over the course of the bioreactor process for IAP produced under all three conditions.

FIG. 12 depicts results from various bioreactor process runs under Condition 1, 2, or 3, on day 12 (Condition 1) or day 13 (Conditions 2 and 3) of the culturing process. Measurements were taken of cell viability, BIAP titer in g/L, percent dimers, AP activity in U/mL, and AP specific activity in U/mg. The results show that the IAP produced from under Condition 3 at day 13 exhibited higher AP activity and AP specific activity on day 13, as compared to IAP produced under Conditions 1 and 2.

FIG. 13 shows results of product quality measurements at 3 L bioreactor scale.

FIG. 14 shows results of product quality measurements at 50 L bioreactor scale.

FIG. 15 shows results of product quality measurements at 200 L bioreactor scale.

FIGS. 16A-D depicts recombinant bIAP titers, viability, viable cell density, and glucose levels for various bioreactor sizes over the culturing period.

FIGS. 17A-D depicts metabolite content for various bioreactor sizes over the culturing period. Measured metabolites include glutamine, glutamate, ammonium, and lactate.

### DETAILED DESCRIPTION OF THE INVENTION

### Overview

Treatment for gastrointestinal (GI) disorders is increasingly looking to the role of the microbiome as a mediator in preserving healthy functioning of the GI tract. As such, the role of alkaline phosphatases (APs) in promoting growth of good bacteria and reversing dysbiosis is a significant and growing field of study in the advancement of treatment options for GI disorders. However, no AP-based drugs have been approved to date.

In particular, intestinal alkaline phosphatase (IAP) is an endogenous protein expressed by the intestinal epithelium that can be used to mitigate inflammation and maintain gut homeostasis. For example, loss of IAP expression or function is associated with increased intestinal inflammation, dysbiosis, bacterial translocation, and systemic inflammation. Its primary functions, among others, in maintaining intestinal homeostasis are generally recognized as the regulation of bicarbonate secretion and duodenal surface pH, long chain fatty acid absorption, mitigation of intestinal inflammation through detoxification of pathogen-associated molecular patterns, and regulation of the gut microbiome. Several substrates that are acted on by IAP's phosphatase functions include lipopolysaccharide (LPS), flagellin, CpG DNA, and nucleotide di- and tri-phosphates. Specifically, IAP is a target for therapeutics due to its ability to inactivate LPS, regulate the microbiome, tighten the gut barrier through enhanced expression of claudins and occludins, and affect metabolism of adenosine tri-phosphate and diphosphate (ATP and ADP).

Providing manufacturing methods capable of producing sufficient IAP with high activity can be difficult to achieve due to the complicated nature of protein biologics.

Accordingly, the present invention provides, inter alia, methods of making IAP in a bioreactor process using cell lines that produce commercially relevant high yields of IAP via adding a supplementary amount of zinc to the cell cultures during the manufacturing process. In various embodiments, the addition of supplementary zinc occurs during a batch feeding step of the manufacturing process, or during a step of the manufacturing process comprising feeding the culture within the bioreactor, or during a purification step of the manufacturing process, or during a formulation step of the manufacturing process.

### Alkaline Phosphatase-Based Agents

The present invention is directed, in part, to pharmaceutical compositions, formulations, uses, and manufacturing methods of one or more alkaline phosphatase-based agents (AP-based agents). Alkaline phosphatases are dimeric metalloenzymes that catalyze the hydrolysis of phosphate esters and dephosphorylate a variety of target substrates at physiological and higher pHs. Alkaline phosphatases are found in prokaryotic as well as in eukaryotic organisms (e.g., in E. coli and mammals). Illustrative AP-based agents that may be utilized in the present invention include, but are not limited to, intestinal alkaline phosphatase (IAP; e.g., human IAP, calf IAP or bovine IAP, chicken IAP, goat IAP), placental alkaline phosphatase (PLAP), placental-like alkaline phosphatase, germ cell alkaline phosphatase (GCAP), tissue non-specific alkaline phosphatase (TNAP; which is primarily found in the liver, kidney, and bone), bone alkaline phosphatase, liver alkaline phosphatase, kidney alkaline phosphatase, bacterial alkaline phosphatase, fungal alkaline phosphatase, shrimp alkaline phosphatase, modified IAP, recombinant IAP, or any polypeptide comprising alkaline phosphatase activity.

In various embodiments, the present invention contemplates the use of alkaline phosphatases derived from eukaryotic or prokaryotic organisms. In some embodiments, the present invention uses mammalian alkaline phosphatases including, but not limited to, intestinal alkaline phosphatase (TAP), bovine intestinal alkaline phosphatase (bIAP), recombinant bovine intestinal alkaline phosphatase (rbIAP), placental alkaline phosphatase (PLAP), germ cell alkaline phosphatase (GCAP), and the tissue non-specific alkaline phosphatase (TNAP).

IAPs

In some embodiments, the AP-based agent is IAP. IAP is produced in the proximal small intestine and is bound to the enterocytes via a glycosyl phosphatidylinositol (GPI) anchor. Some IAP is released into the intestinal lumen in conjunction with vesicles shed by the cells and as soluble protein stripped from the cells via phospholipases. The enzyme then traverses the small and large intestine such that some active enzyme can be detected in the feces. In an embodiment, the IAP is human IAP (hIAP). In an embodiment, the IAP is calf IAP (cIAP), also known as bovine IAP (bIAP). There are multiple isozymes of bIAP, for example, with bIAP II and IV having higher specific activity than bIAP I. In an embodiment, the IAP is any one of the cIAP or bIAP isozymes (e.g., bIAP I, II, and IV). In an embodiment, the IAP is bIAP II. In another embodiment, the IAP is bIAP IV.

IAP Variants

Also included within the definition of IAPB are IAP variants. An IAP variant has at least one or more amino acid modifications, generally amino acid substitutions, as compared to the parental wild-type sequence. In some embodiments, an IAP of the invention comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein. In addition, IAP variants retain most or all of their biochemical activity, measured as described herein.

Mammalian alkaline phosphatases are GPI anchored proteins. They have signal peptides and are translated into the secretory pathway. Once in the endoplasmic reticulum (ER), the proteins are glycosylated and folded. There are two disulfide bonds as well as a single free cysteine that is apparently not accessible on the surface. In the late ER, the carboxy terminus is removed and the GPI anchor is appended. GPI anchoring is therefore a process that occurs at the carboxy terminus of the alkaline phosphatase. The inclusion of stop codons at the anchor site enables secretion of biologically active protein (presumably the homodimer). While there is no consensus sequence, the carboxy terminus includes three amino acids, termed omega, omega +1, and omega +2 which are followed by a short stretch of hydrophilic amino acids and then a stretch of hydrophobic amino acids. Without wishing to be bound by theory, it is believed that the hydrophobicity is critical for embedding the carboxy terminus in the ER membrane. There, an enzymatic reaction replaces the carboxy terminus with the GPI anchor.

Within human placental alkaline phosphatase (hPLAP), the GPI anchor is attached at an aspartate (D) in the sequence, DAAH. Similarly, hIAP, bIAP II, and bIAP IV also have this DAAH sequence conserved, potentially serving as the GPI anchor site. Mutational studies with hPLAP indicate that preventing GPI anchoring results in intracellular retention. In addition, mutations around the anchor site or in the hydrophobic domain either 1) prevent anchor attachment leading to intracellular retention or 2) do not block anchor attachment. Without wishing to be bound by theory, it is believed that the hydrophobic domain serves as a signal for GPI anchor attachment. Truncating or eliminating the hydrophobic domain leads to secretion. Finally, there is a single mutation in the hydrophobic domain that, in hPLAP, enables secretion of the protein with its hydrophobic domain intact.

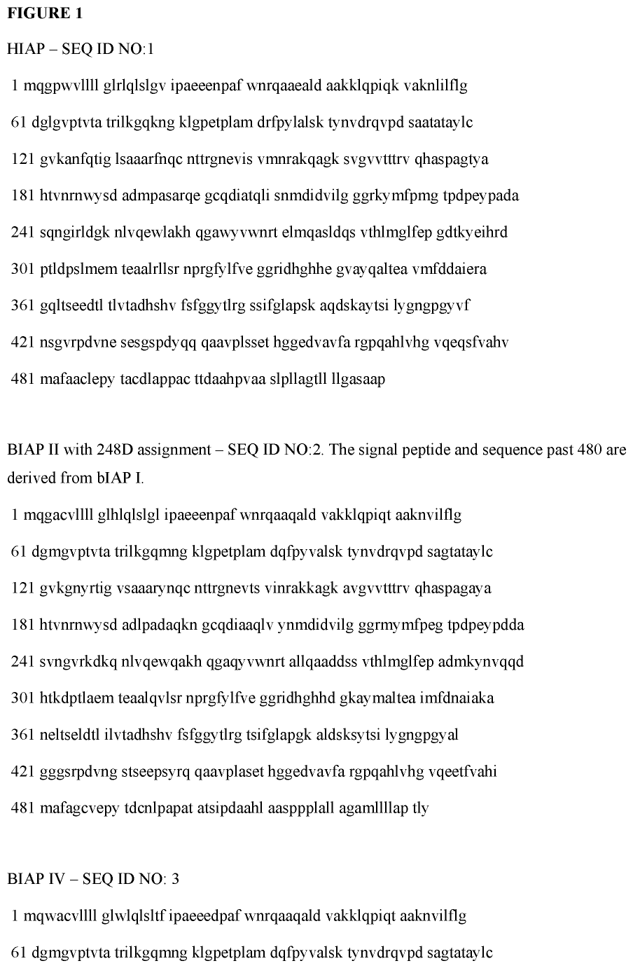

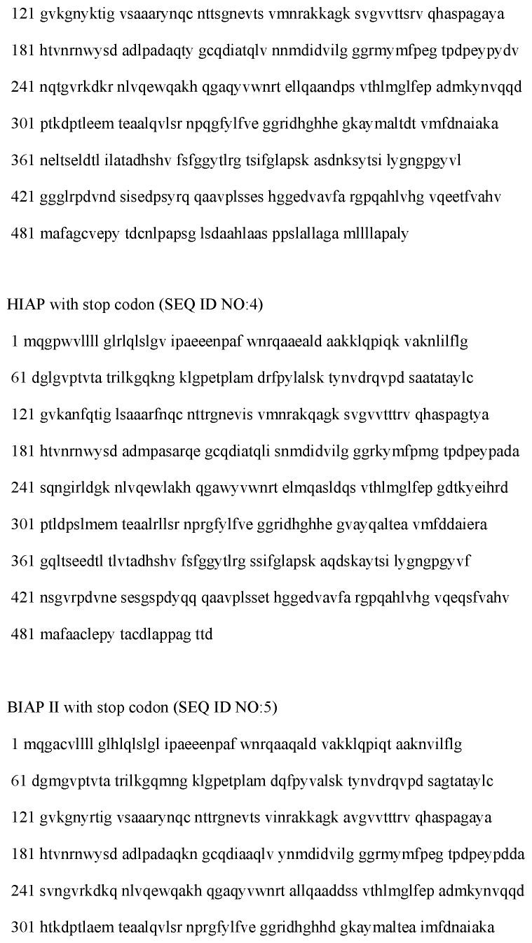

In other embodiments, the AP-based agent of the invention is a secreted protein; that is, in some embodiments, the AP-based agent is not GPI anchored, leading to secretion rather than intracellular retention. This can be accomplished in several ways. In some embodiments, the AP-based agent may lack the GPI anchor site, e.g. have the DAAH site removed, leading to secretion. Alternatively, this can be accomplished in some embodiments, the AP-based agent comprises a stop codon that is inserted immediately before the GPI anchor site. In an embodiment, the AP-based agent comprises a stop codon after the aspartate in the DAAH consensus site (e.g., at amino acid 503 of hIAP and bIAP IV or amino acid 506 of bIAP II). FIG. 1 depicts HIAP with a stop codon (SEQ ID NO:4), bIAP II with a stop codon (SEQ ID NO:5), and bIAP IV with a stop codon (SEQ ID NO:6). In an embodiment, the AP-based agent is bIAP IV and includes a stop codon after amino acid 508 to mimic a secreted PLAP construct as depicted in FIG. 1 (SEQ ID NO:7).

Human IAP

In various embodiments, the AP-based agent is a hIAP. In some embodiments, the AP-based agent is hIAP comprising the amino acid sequence of SEQ ID NO:1 as depicted in FIG. 1 or a variant as described herein, as long as the hIAP variant retains at least 80, 85, 90, 95, 98 or 100% of the phosphatase activity as compared to the wild type enzyme using an assay as outlined herein.

Included within the definition of hIAP are amino acid modifications, with amino acid substitutions finding particular use in the present invention. For example, without wishing to be bound by theory, it is believed that a cysteine at the carboxy terminus of the AP-based agent (e.g., at position 500 of SEQ ID NO:1) may interfere with protein folding. Accordingly, in some embodiments, the AP-based agent includes a mutation of the cysteine (e.g., at position 500 of SEQ ID NO:1). In some embodiments, the cysteine is replaced with any amino acid, although glycine finds particular use in some embodiments. Furthermore, the C-terminal cysteine can also be deleted.

As will be appreciated by those skilled in the art, additional amino acid modifications can be made in hIAP as discussed herein. For example, in some embodiments, a stop codon may be inserted after the aspartate in the DAAH consensus site (e.g., at amino acid 503 of hIAP). FIG. 1 depicts hIAP with an inserted stop codon (SEQ ID NO:4).

Bovine IAPs

In some embodiments, the IAP is a bovine IAP (bIAP).

bIAP II

In various embodiments, the AP-based agent is bovine IAP II (bIAP II) or a variant as described herein, as long as the bIAP variant retains at least 80, 85, 90, 95, 98 or 100% of the phosphatase activity using an assay as outlined herein. In an embodiment, the bIAP II comprises the signal peptide and carboxy terminus of bIAP I. In an embodiment, the bIAP II comprises an aspartate at position 248 (similar to bIAP IV). In an embodiment, the bIAP II comprises the amino acid sequence of SEQ ID NO:2. FIG. 1 depicts BIAP II with 248D assignment—SEQ ID NO:2. The signal peptide and sequence past 480 are derived from bIAP I.

Also included within the definition of bIAP II are amino acid variants as described herein. For example, in some embodiments, a stop codon may be inserted after the aspartate in the DAAH consensus site (e.g., at amino acid 506 of bIAP II). FIG. 1 depicts bIAP II with an inserted stop codon (SEQ ID NO:5).

bIAP IV

In various embodiments, the AP-based agent is bIAP IV or a variant thereof as described herein, as long as the bIAP IV variant retains at least 80, 85, 90, 95, 98 or 100% of the phosphatase activity using an assay as outlined herein. In an embodiment, the bIAP IV comprises the amino acid sequence of SEQ ID NO:3, as depicted in FIG. 1.

Also included within the definition of bIAP IV are amino acid variants as described herein. For example, in some embodiments, a stop codon may be inserted after the aspartate in the DAAH consensus site (e.g., at amino acid 503 of bIAP IV). FIG. 1 depicts bIAP IV with an inserted stop codon (SEQ ID NO:6). In an embodiment, the AP-based agent is bIAP IV and includes a stop codon after amino acid 508 to mimic a secreted PLAP construct, as depicted in FIG. 1 (SEQ ID NO:7).

Bacterial APs

In various embodiments, the present invention contemplates the use of bacterial alkaline phosphatases. In some embodiments, the AP-based agent of the invention is derived from Bacillus subtilis. Bacillus subtilis is a Gram-positive bacterium found in soil and the GI tract of humans. Bacillus subtilis secretes high levels of proteins into the environment and in the human GI tract that are properly folded. Without wishing to be bound by theory, it is believed that Bacillus subtilis secreted proteins in the GI tract may be resistant to degradation by common GI proteases. Bacillus subtilis expresses at high levels an alkaline phosphatase multigene family. Among those isozymes, alkaline phosphatase IV is responsible for the majority of total alkaline phosphatase expression and activity in B. subtilis. In some embodiments, the AP-based agent of the invention is derived from Bacillus licheniformis. In some embodiments, the AP-based agent of the invention is derived from Escherichia coli.

Accordingly, in an illustrative embodiment, the AP-based agent of the invention is derived from alkaline phosphatase IV of Bacillus subtilis. In an embodiment, the bacterial alkaline phosphatase may have nucleotide and amino acid sequences as depicted in FIG. 1, including Bacillus subtilis JH642 alkaline phosphatase IV, mature protein nucleotide sequence—SEQ ID NO: 16; and Bacillus subtilis JH642 alkaline phosphatase IV, mature protein amino acid sequence—SEQ ID NO: 17, or variants as described herein, as long as the hIAP variant retains at least 80, 85, 90, 95, 98 or 100% of the phosphatase activity using an assay as outlined herein.

In some embodiments, the AP-based agents include bacterial alkaline phosphatases that have one or more mutations that alter catalytic activity. In some embodiments, the bacterial alkaline phosphatases include one or more mutations such that their catalytic activity is similar or higher than mammalian alkaline phosphatases. In some embodiments, the bacterial alkaline phosphatases include one or more mutations that alter their de-phosphorylation profile. In an embodiment, the bacterial alkaline phosphatases of the invention exhibit similar de-phosphorylation profile as mammalian alkaline phosphatases. In some embodiments, the bacterial alkaline phosphatases include one or more mutations that alter their activity at higher pH. In an embodiment, the bacterial alkaline phosphatases of the invention exhibit similar activity at higher pH as mammalian alkaline phosphatases. In some embodiments, the bacterial alkaline phosphatases include one or more mutations that alter their metal requirements. In an embodiment, the bacterial alkaline phosphatases of the invention exhibit metal requirements (e.g., Mg) similar to mammalian alkaline phosphatases.

For example, in certain embodiments, the AP-based agent of the invention is derived from Bacillus subtilis JH642 alkaline phosphatase IV, and has one or more mutations at positions 101, 328, 330, and 374. For example, the AP-based agent may include one or more of the following mutations: D101A, W328H, A330N and G374C.

Fusion Proteins

In some embodiments, the AP-based agent comprises an alkaline phosphatase fused to a “fusion partner”, which is a protein domain that is added either to the N- or C-terminus of the IAP domain, optionally including a linker. In some embodiments, the alkaline phosphatase is fused to a protein domain that promotes protein folding and/or protein purification and/or protein dimerization and/or protein stability. In various embodiments, the AP-based agent fusion protein has an extended serum half-life. In various embodiments, the AP-based agent of the invention is an Fc fusion protein.

In an embodiment, the alkaline phosphatase is fused to an immunoglobulin Fc domain and/or hinge region. In an embodiment, the AP-based agent of the invention comprises an alkaline phosphatase fused to the hinge region and/or Fc domain of IgG.

In various embodiments, the AP-based agent is fused to a Fc domain of IgG comprising one or more mutations. In some embodiments, the one or more mutations in the Fc domain of IgG function to increase serum half-life and longevity. In some embodiments, the Fc domain of IgG comprises one or more mutations at amino acid residues 251-256, 285-290, 308-314, 385-389 and 428-436, numbered according to the EU index as in Kabat (see Kabat et al., (1991) Sequences of Proteins of Immunological Interest, U.S. Public Health Service, National Institutes of Health, Washington, D.C.). In some embodiments, at least one of the amino acid substitutions in the Fc domain of IgG is at amino acid residue 252, 254, 256, 309, 311, 433 or 434. In an embodiment, the amino acid substitution at amino acid residue 252 is a substitution with tyrosine, phenylalanine, tryptophan or threonine. In an embodiment, the amino acid substitution at amino acid residue 254 is a substitution with threonine. In an embodiment, the amino acid substitution at amino acid residue 256 is a substitution with serine, arginine, glutamine, glutamic acid, aspartic acid, or threonine. In an embodiment, the amino acid substitution at amino acid residue 309 is a substitution with proline. In an embodiment, the amino acid substitution at amino acid residue 311 is a substitution with serine. In an embodiment, the amino acid substitution at amino acid residue 385 is a substitution with arginine, aspartic acid, serine, threonine, histidine, lysine, alanine or glycine. In an embodiment, the amino acid substitution at amino acid residue 386 is a substitution with threonine, proline, aspartic acid, serine, lysine, arginine, isoleucine, or methionine. In an embodiment, the amino acid substitution at amino acid residue 387 is a substitution with arginine, proline, histidine, serine, threonine, or alanine. In an embodiment, the amino acid substitution at amino acid residue 389 is a substitution with proline, serine or asparagine. In an embodiment, the amino acid substitution at amino acid residue 433 is a substitution with arginine, serine, isoleucine, proline, or glutamine. In an embodiment, the amino acid substitution at amino acid residue 434 is a substitution with histidine, phenylalanine, or tyrosine.

In some embodiments, the Fc domain of IgG comprises one or more mutations at amino acid residue 252, 254, 256, 433, 434, or 436. In an embodiment, the Fc domain of IgG includes a triple M252Y/S254T/T256E mutation or YTE mutation. In another embodiment, the Fc domain of IgG includes a triple H433K/N434F/Y436H mutation or KFH mutation. In a further embodiment, the Fc domain of IgG includes a YTE and KFH mutation in combination. Additional illustrative mutations in the Fc domain of IgG are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, and U.S. Pat. No. 7,083,784, the entire contents of which are hereby incorporated by reference. In various embodiments, the one or more mutations in the Fc domain of IgG increases affinity for the neonatal Fc receptor (FcRn). In some embodiments, the one or more mutations in the Fc domain of IgG increases affinity for FcRn at a pH of about 6.0, about 6.1, about 6.2, about 6.3, about 6.4, or about 6.5.

In various embodiments, the alkaline phosphatase is fused to one or more of PEG, XTENylation (e.g. as rPEG), polysialic acid (POLYXEN), albumin, elastin-like protein, elastin like protein (ELP), PAS, HAP, GLK, CTP, and transferrin. In various embodiments, the alkaline phosphatase is fused to one or more of the agents described in BioDrugs (2015) 29:215-239, the entire contents of which are hereby incorporated by reference.

Linkers

In an embodiment, the alkaline phosphatase is fused to a protein domain (e.g., an immunoglobulin Fc domain) via a linker to the GPI anchor site. For example, the alkaline phosphatase may be fused to a protein domain via the aspartate at the GPI anchor sequence. The invention contemplates the use of a variety of linker sequences. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et al., (2000), Protein Eng. 13(5):309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the present AP-based agent. In another example, the linker may function to target the AP-based agent to a particular cell type or location.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). In an embodiment, the linker sequence is GGSGGSGGGGSGGGGS. Additional illustrative linkers include, but are not limited to, linkers having the sequence LE, GGGGS, (GGGGS)n (n=1-4), (Gly)8, (Gly)6, (EAAAK)n (n=1-3), A(EAAAK)nA (n =2-5), AEAAAKEAAAKA, A(EAAAK)4ALEA(EAAAK)4A, PAPAP, KESGSVS SEQLAQFRSLD, EGKSSGSGSESKST, GSAGSAAGSGEF, and (XP)n, with X designating any amino acid, e.g., Ala, Lys, or Glu. In some embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). In some embodiments, the linker is a synthetic linker such as PEG.

Illustrative Fc fusion constructs of the invention include those depicted in FIG. 1, including BIAP II with Fc Fusion (SEQ ID NO:8)—Fc domain underlined; and BIAP IV with Fc Fusion (SEQ ID NO:9)—Fc domain underlined.

Pro-Enzyme Fusions

The invention additionally provides C-terminal fusions for pro-enzyme functions. Without wishing to be bound by theory, it is believed that mammalian alkaline phosphatases may also be generated as inactive pro-enzymes. This is because alkaline phosphatases can dephosphorylate ATP, so that activity in the ER could drain the ER of its major energy source. Without wishing to be bound by theory, it is believed that the inhibitory function is located to the carboxy terminus that would be relieved upon GPI anchor addition. Alternatively, other activities such as folding or metal (Zn or Mg) inclusion could control activity.

In various embodiments, the AP-based agent of the invention is a pro-enzyme. In an embodiment, the activity of the proenzyme is suppressed by a carboxy terminus. In an embodiment, protease removal of the carboxy terminus reactivates the enzymatic activity of the alkaline phosphatase. In an embodiment, the pro-enzyme is more efficiently secreted than the enzyme without the carboxy terminus.

A Saccharomyces alkaline phosphatase, Pho8, is produced as an inactive pro-enzyme. It is not GPI anchored, but is a transmembrane protein with its amino terminus extending out of a lysosome into the cytoplasm. Within the lysosome, an enzyme, PEP4, cleaves the carboxy terminus to activate the enzyme.

In some embodiments, for generation of the pro-enzyme, the native carboxy terminus of the alkaline phosphatase is replaced with the analogous sequence from hPLAP. In some embodiments, a mutation is made in the hydrophobic carboxy tail to promote protein secretion without cleavage of the carboxy terminus. In an illustrative embodiment, a single point mutation such as a substitution of leucine with e.g., arginine is generated in the hydrophobic carboxy terminus (e.g. ALLPLLAGTL is changed to e.g., ALLPLRAGTL) to result in secretion of the enzyme without removal of the carboxy terminus.

In an embodiment, the AP-based agent is altered to include a specific enzyme cleavage site which allows subsequent removal of the carboxy terminus. In an embodiment, the AP-based agent includes a protease cleavage site. Illustrative protease cleavage sites include, but are not limited to, cleavage sites recognized by furin, Rhinovirus 16 3C protease, factor Xa protease, trpysin, chymotrypsin, elastase, pepsin, papain subtilisin, thermolysin, V-8 protease, submaxillaris protease, clostripain, thrombin, collagenase, and any other endoproteases. In an alternative embodiment, the AP-based agent includes a cleavage site recognized by a digestive enzyme present in the GI tract. In such embodiments, the AP-based agent may be administered as a pro-drug that is subsequently activated in the GI tract.

In an illustrative embodiment, the proenzyme is a proenzyme of bIAP IV having sequences depicted in FIG. 1, including BIAP IV with the hPLAP Carboxy Terminus and Mutation for Unprocessed Secretion and RV3C Cleavage (at . . . LEVLFQGP . . . ) (SEQ ID NO:10); and BIAP IV with hPLAP Carboxy Terminus and Mutation for Unprocessed Secretion and FXa Cleavage (at . . . IEGR . . . ) (SEQ ID NO:11).

Expression Variants

In various embodiments, the AP-based agent of the invention is efficiently expressed and secreted from a host cell. In an embodiment, the AP-based agent of the invention is efficiently transcribed in a host cell. In another embodiment, the AP-based agent exhibits enhanced RNA stability and/or transport in a host cell. In another embodiment, the AP-based agent is efficiently translated in a host cell. In another embodiment, the AP-based agent exhibits enhanced protein stability.

In various embodiments, the AP-based agents are efficiently expressed in a host cell. In an embodiment, the Kozak sequence of the DNA construct encoding the AP-based agent is optimized. The Kozak sequence is the nucleotide sequence flanking the ATG start codon that instructs the ribosome to start translation. There is flexibility in the design of a Kozak sequence, but one canonical sequence is GCCGCCACCATGG. The purine in the −3 position and the G in the +4 position are the most important bases for translation initiation. For hIAP, bIAP II, and bIAP IV, the second amino acid, that is, the one after the initiator methionine, is glutamine. Codons for glutamine all have a C in the first position. Thus, their Kozak sequences all have an ATGC sequence. Accordingly, in various embodiments, the ATGC sequence is changed to ATGG. This can be achieved by changing the second amino acid to a glycine, alanine, valine, aspartate, or glutamic acid, all of whose codons have a G in the first position. These amino acids may be compatible with signal peptide function. In alternative embodiments, the entire signal peptide is substituted for peptide having a canonical Kozak sequence and is derived from a highly expressed protein such as an immunoglobulin.

In various embodiments, the signal peptide of the AP-based agent may be deleted and/or substituted. For example, the signal peptide may be deleted, mutated, and/or substituted (e.g., with another signal peptide) to ensure optimal protein expression.

In some embodiments, the DNA construct encoding the AP-based agent of the invention comprises untranslated DNA sequences. Such sequences include an intron, which may be heterologous to the IAP protein or native to the IAP protein including the native first and/or second intron and/or a native 3′ UTR. Without wishing to be bound by theory, it is believed that inclusion of these sequences enhance protein expression by stabilizing the mRNA. Accordingly, in various embodiments, the DNA construct encoding the AP-based agent of the invention comprises the 5′UTR and/or the 3′UTR. Provided in FIG. 1 are illustrative IAP DNA sequences with a first intron and a 3′UTR, including hIAP with native first intron (shown as bolded and underlined)—SEQ ID NO: 12; hIAP with native 3′ UTR (shown as bolded and underlined)—SEQ ID NO: 13; bIAP IV with the first intron from bIAP I (shown as bolded and underlined)—SEQ ID NO: 14; and bIAP IV with the 3′ UTR from bIAP I (shown as bolded and underlined)—SEQ ID NO: 15.

In various embodiments, the AP-based agent of the invention comprises a nucleotide sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein.

In various embodiments, the AP-based agent of the invention may comprise an amino acid sequence having one or more amino acid mutations relative to any of the protein sequences described herein. In some embodiments, the one or more amino acid mutations may be independently selected from substitutions, insertions, deletions, and truncations.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, a-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general).

Mutations may be made to the AP-based agent of the invention to select for agents with desired characteristics. For examples, mutations may be made to generate AP-based agents with enhanced catalytic activity or protein stability. In various embodiments, directed evolution may be utilized to generate AP-based agents of the invention. For example, error-prone PCR and DNA shuffling may be used to identify mutations in the bacterial alkaline phosphatases that confer enhanced activity and/or stability.

### Manufacturing Process

Cell-Line Expression

In one aspect, the present invention provides methods for manufacturing AP-based agents disclosed herein in cell lines, including mammalian cell lines. As used herein, the term “host cells” refers to cells that can be used to produce AP-based agents disclosed herein. In some aspects of the present invention, AP-based agents are produced in non-recombinant expression systems. In various embodiments, the present invention contemplates use of a protein source that produces an AP-based agent. In some embodiments, the AP-based agent is not produced in a cell.

Various cell lines may be used to express recombinant polypeptides and proteins, such as the AP-based agents described herein. In some embodiments, mammalian cell lines are used. Expression of recombinant proteins in mammalian cells may be desirable because these proteins are generally correctly folded, appropriately modified, and biologically functional. Examples of suitable mammalian host cell lines include, but are not limited to, COS-1 or COS-7 (monkey kidney-derived), L-929 (murine fibroblast-derived), C127 (murine mammary tumor-derived), 3T3 (murine fibroblast- derived), CHO (Chinese hamster ovary-derived; including DHFR CHO (Urlaub et al., Proc. Natl. Acad. Sci. USA 77:4216 (1980)), HeLa (human cervical cancer-derived), BHK (hamster kidney fibroblast-derived, e.g., BHK21), PER.C6 (human embryonic retinal cells), and HEK-293 (human embryonic kidney-derived) cell lines and variants thereof. In some embodiments, the mammalian host cell lines are monkey kidney CV1 line transformed by SV40 (COS-7); human embryonic kidney line (293 or 293 cells as described, e.g., in Graham et al., Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); HKB11 cells (a somatic cell fusion between human kidney and human B cells as described in example, U.S. Pat. No. 6,136,599); mouse mammary tumor cells (MMT 060562); TRI cells (as described, e.g., in Mather et al., Annals N Y. Acad. Sci. 383:44-68 (1982)); MRC 5 cells; FRhL-2 cells, NIH3T3 cell, Jurkat, and FS4 cells. In some embodiments, the mammalian cell line used for the present invention is CHO cells. In some embodiments, the mammalian cell line used for the present invention is a human amniotic cell line.

As used herein, the term “average cell productivity” refers to the average amount of AP-based protein (in picograms) produced per cell per day of the culturing process. In some embodiments, host cells are able to produce AP-based agents in an amount of or greater than 20 picogram/cell/day, 25 picogram/cell/day, 30 picogram/cell/day, 35 picogram/cell/day, 40 picogram/cell/day, 45 picogram/cell/day, or 50 picogram/cell/day, 55 picogram/cell/day, 60 picogram/cell/day, 65 picogram/cell/day, 70 picogram/cell/day, 75 picogram/cell/day, 80 picogram/cell/day, 85 picogram/cell/day, 90 picogram/cell/day, 95 picogram/cell/day, or 100 picogram/cell/day, on average. In certain embodiments, the host cells involved in the process of the present invention have an average cell productivity of at least 35 picogram/cell/day.

Vectors and Nucleic Acid Constructs

Various nucleic acid constructs can be used to express high levels of AP-based agents described herein in host cells. A suitable expression vector construct typically includes, in addition to nucleic AP-encoding sequences, regulatory sequences, gene control sequences, strong transcription promoters, transcription and/or translation terminators, ribosome binding sites for translational initiation, and/or other appropriate sequences for expression of the protein and, optionally, for replication of the construct. Typically, the coding region is operably linked with one or more of these nucleic acid components.

Regulatory Sequences

Expression control regions are regulatory polynucleotides (sometimes referred to herein as elements), such as promoters and enhancers, that influence expression of an operably linked nucleic acid. For example, mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Culture Medium

Typically, medium solutions provide, without limitation, essential and nonessential amino acids, vitamins, energy sources, lipids, and trace elements required by the cell for at least minimal growth and/or survival. In other embodiments, the medium may contain an amino acid(s) derived from any source or method known in the art, including, but not limited to, an amino acid(s) derived either from single amino acid addition(s) or from a peptone or protein hydrolysate addition(s) (including animal or plant source(s)). Vitamins such as, but not limited to, Biotin, Pantothenate, Choline Chloride, Folic Acid, Myo-Inositol, Niacinamide, Pyridoxine, Riboflavin, Vitamin B12, Thiamine, Putrescine and/or combinations thereof. Salts such as, but not limited to, ZnSO₄, CaCl₂, KCl, MgCl₂, NaCl, Sodium Phosphate Monobasic, Sodium Phosphate Dibasic, Sodium Selenite, CuSO₄, ZnCl₂, and/or combinations thereof. Fatty acids such as, but not limited to, Arachidonic Acid, Linoleic Acid, Oleic Acid, Lauric Acid, Myristic Acid, as well as Methyl-beta-Cyclodextrin and/or combinations thereof. In some embodiments, medium comprises additional components such as glucose, glutamine, Na-pyruvate, insulin or ethanolamine, a protective agent such as Pluronic F68. In some embodiments, the medium may also contain components that enhance growth and/or survival above the minimal rate, including hormones and growth factors. Medium may also comprise one or more buffering agents. The buffering agents may be designed and/or selected to maintain the culture at a particular pH (e.g., a physiological pH, (e.g., pH 6.8 to pH 7.4)). A variety of buffers suitable for culturing cells are known in the art and may be used in the methods. Suitable buffers (e.g., bicarbonate buffers, HEPES buffer, Good's buffers, etc.) are those that have the capacity and efficiency for maintaining physiological pH despite changes in carbon dioxide concentration associated with cellular respiration. The solution is preferably formulated to a pH and salt concentration optimal for cell survival and proliferation.

In some embodiments, the culture medium comprises, but is not limited to, glutamine, a mixture of sodium hypoxanthine and thymidine (HT), ZnSO₄, MgCl₂, a poloxamer (e.g., Kolliphor P188), and antifoam.

In some embodiments, the manufacturing culturing process includes a step of adding a quantity of supplemental zinc to the culture medium. In some embodiments, the supplemental zinc is a zinc carrier including, but not limited to, ZnSO₄, ZnCl₂, ZnBr₂, zinc citrate, hydrolysate, and plasma zinc bound to serum albumin. In certain embodiments, the step of adding a quantity of supplemental zinc to the culture medium occurs about 2 days, or about 3 days, or about 4 days, or about 5 days, or about 6 days, or about 7 days, or about 8 days, or about 9 days, or about 10 days, or about 11 days, or about 12 days, or about 13 days after initiation of the culturing process (e.g., the day the cells are seeded in the bioreactor to begin the culturing process). In some embodiments, the step of adding a quantity of supplemental zinc to the culture medium occurs at least once, at least twice, at least three times, at least four times, or at least five times during the culturing process. In some embodiments, the quantity of supplemental zinc is between about 50 to 110 μM zinc, between about 60 to 100 μM zinc, between about 70 to 90 μM zinc, between about 80 to 90 μM zinc, or between about 50-500 μM zinc. In some embodiments, the quantity of supplemental zinc is at least 50 μM zinc, or at least 60 μM zinc, or at least 70 μM zinc, or at least 80 μM zinc, or at least 90 μM zinc, or at least 100 μM zinc, or at least 110 μM zinc, or at least 120 μM zinc, or at least 130 μM zinc, or at least 140 μM zinc, or at least 150 μM zinc, or at least 160 μM zinc, or at least 170 μM zinc, or at least 180 μM zinc, or at least 190 μM zinc, or at least 200 μM zinc, or at least 210 μM zinc, or at least 220 μM zinc, or at least 230 μM zinc, or at least 240 μM zinc, or at least 250 μM zinc, or at least 260 μM zinc, or at least 270 μM zinc, or at least 280 μM zinc, or at least 290 μM zinc, or at least 300 μM zinc, or at least 310 μM zinc, or at least 320 μM zinc, or at least 330 μM zinc, or at least 340 μM zinc, or at least 350 μM zinc, or at least 360 μM zinc, or at least 370 μM zinc, or at least 380 μM zinc, or at least 390 μM zinc, or at least 400 μM zinc, or at least 410 μM zinc, or at least 420 μM zinc, or at least 430 μM zinc, or at least 440 μM zinc, or at least 450 μM zinc, or at least 460 μM zinc, or at least 470 μM zinc, or at least 480 μM zinc, or at least 490 μM zinc, or at least 500 μM zinc. In some embodiments, the quantity of supplemental zinc is about 50 μM zinc, or about 60 μM zinc, or about 70 μM zinc, or about 80 μM zinc, or about 90 μM zinc, or about 100 μM zinc, or about 110 μM zinc, or about 120 μM zinc, or about 130 μM zinc, or about 140 μM zinc, or about 150 μM zinc, or about 160 μM zinc, or about 170 μM zinc, or about 180 μM zinc, or about 190 μM zinc, or about 200 μM zinc, or about 210 μM zinc, or about 220 μM zinc, or about 230 μM zinc, or about 240 μM zinc, or about 250 μM zinc, or about 260 μM zinc, or about 270 μM zinc, or about 280 μM zinc, or about 290 μM zinc, or about 300 μM zinc, or about 310 μM zinc, or about 320 μM zinc, or about 330 μM zinc, or about 340 μM zinc, or about 350 μM zinc, or about 360 μM zinc, or about 370 μM zinc, or about 380 μM zinc, or about 390 μM zinc, or about 400 μM zinc, or about 410 μM zinc, or about 420 μM zinc, or about 430 μM zinc, or about 440 μM zinc, or about 450 μM zinc, or about 460 μM zinc, or about 470 μM zinc, or about 480 μM zinc, or about 490 μM zinc, or about 500 μM zinc. In specific embodiments, the quantity of supplemental zinc is 80 μM zinc.

Culture Conditions

The present invention provides a method of producing AP-based agents at small and large scale. Procedures for producing AP-based agents of interest may include batch cultures and fed-batch cultures. Batch culture processes comprise inoculating a production culture with a seed culture of a particular cell density, growing the cells under conditions (e.g., suitable culture medium, pH, and temperature) conducive to cell growth, viability, and/or productivity, harvesting and/or separating the culture when the cells reach a specified cell density, and purifying the expressed polypeptide. Fed-batch culture procedures include an additional step or steps of supplementing the batch culture with nutrients and other components that are consumed during the growth of the cells. In some embodiments, a production method according to the present invention uses a fed-batch culture system.

Accordingly, in some embodiments, the production culture is fed-batch with one or more feeds. For example, in certain embodiments, the production method of the present invention includes a step of batch feeding at a discrete time point over the course of the culturing process. In certain embodiments, the batch feeding occurs at least once every day over the course of the culturing process. In some embodiments, the batch feeding occurs for a discrete time period over the course of the culturing process. In specific embodiments, the batch feeding is initiated at least 2 days after initiation of the culturing process (e.g., the day the cells are seeded in the bioreactor to begin the culturing process). In specific embodiments, the batch feeding is terminated at least 10 days, or at least 12 days, or at least 13 days after initiation of the culturing process.

In some embodiments, the batch feeding comprises one or more feeds. In some embodiments, the batch feeding comprises at least two separate feeds. For example, in certain embodiments, a feed comprises ingredients including, but not limited to, a carbon source, concentrated amino acids, vitamins, salts, and/or trace minerals. A feed can further comprise at least 40 g/L glucose, or at least 50 g/L glucose, or at least 60 g/L glucose, or at least 70 g/L glucose. In certain embodiments, a feed comprises at least 5 mg/L insulin, or at least 6 mg/L insulin, or at least 7 mg/L insulin, or at least 8 mg/L insulin, or at least 9 mg/L insulin, or at least 10 mg/L insulin, or at least 11 mg/L insulin, or at least 12 mg/L insulin, or at least 13 mg/L insulin, or at least 14 mg/L insulin, or at least 15 mg/L insulin. In some embodiments, the feeding of the first feed comprises about 2.0% of the volume in the bioreactor, or about 2.1% of the volume in the bioreactor, or about 2.2% of the volume in the bioreactor, or about 2.3% of the volume in the bioreactor, or about 2.4% of the volume in the bioreactor, or about 2.5% of the volume in the bioreactor, or about 2.6% of the volume in the bioreactor, or about 2.7% of the volume in the bioreactor, or about 2.8% of the volume in the bioreactor, or about 2.9% of the volume in the bioreactor, or about 3.0% of the volume in the bioreactor, or about 3.1% of the volume in the bioreactor, or about 3.2% of the volume in the bioreactor, or about 3.3% of the volume in the bioreactor, or about 3.4% of the volume in the bioreactor, or about 3.5% of the volume in the bioreactor.

In specific embodiments, a second feed comprises ingredients including, but not limited to, a carbon source, concentrated amino acids, vitamins, salts, and/or trace minerals. In some embodiments, the feeding of the second feed comprises about 0.20% of the volume in the bioreactor, or about 0.21% of the volume in the bioreactor, or about 0.22% of the volume in the bioreactor, or about 0.23% of the volume in the bioreactor, or about 0.24% of the volume in the bioreactor, or about 0.25% of the volume in the bioreactor, or about 0.26% of the volume in the bioreactor, or about 0.27% of the volume in the bioreactor, or about 0.28% of the volume in the bioreactor, or about 0.29% of the volume in the bioreactor, or about 0.30% of the volume in the bioreactor, or about 0.31% of the volume in the bioreactor, or about 0.32% of the volume in the bioreactor, or about 0.33% of the volume in the bioreactor, or about 0.34% of the volume in the bioreactor, or about 0.35% of the volume in the bioreactor.

In some embodiments, the ratio of feeding of the first feed to the second feed is about 15:1, or about 14:1, or about 13:1, or about 12:1, or about 11:1, or about 10:1, or about 9:1, or about 8:1, or about 7:1, or about 6:1, or about 5:1. In certain embodiments, the ratio of feeding of the first feed to the second feed is about 10:1.

In some embodiments, at least one, at least two, at least three, at least four or at least five temperature shifts occur during the culturing process. In some embodiments, a first temperature shift occurs at about 24, about 36, about 48, about 60, about 72, about 84, about 96 or about 108 hours after initiation of the culturing process (e.g., the day the cells are seeded in the bioreactor to begin the culturing process). In some embodiments, a second temperature shift occurs at about 240 hours, about 252 hours, about 264 hours, about 276 hours, about 288 hours, about 300 hours, about 312 hours, or about 324 hours after initiation of the culturing process (e.g., the day the cells are seeded in the bioreactor to begin the culturing process). In some embodiments, a first temperature shift occurs between 24 and 36 hours, between about 24 and 48 hours, between about 24 and 60 hours, between about 24 and 72 hours, between about 24 and 84 hours, between about 24 and 96 hours, between about 24 and 108 hours, between about 36 and 48 hours, between about 36 and 60 hours, between about 36 and 72 hours, between about 36 and 84 hours, between about 36 and 96 hours, between about 36 and 108 hours, between about 48 and 60 hours, between about 48 and 72 hours, between about 48 and 84 hours, between about 48 and 96 hours, between about 48 and 108 hours, between about 60 and 72 hours, between about 60 and 84 hours, between about 60 and 96 hours, between about 60 and 108 hours, between about 72 and 84 hours, between about 72 and 96 hours, between about 72 and 108 hours, between about 84 and 96 hours, between about 84 and 108 hours, or between about 96 and 108 hours after initiation of the culturing process (e.g., the day the cells are seeded in the bioreactor to begin the culturing process). In some embodiments, a second temperature shift occurs between about 240 and 252 hours, between about 240 and 264 hours, between about 240 and 276 hours, between about 240 and 288 hours, between about 240 and 300 hours, between about 240 and 312 hours, between about 240 and 324 hours, between about 252 and 264 hours, between about 252 and 276 hours, between about 252 and 288 hours, between about 252 and 300 hours, between about 252 and 312 hours, between about 252 and 324 hours, between about 264 and 276 hours, between about 264 and 288 hours, between about 264 and 300 hours, between about 264 and 312 hours, between about 264 and 324 hours, between about 276 and 288 hours, between about 276 and 300 hours, between about 276 and 312 hours, between about 276 and 324 hours, between about 288 and 300 hours, between about 288 and 312 hours, between about 288 and 324 hours, between about 300 and 312 hours, between about 300 and 324, or between about 312 and 324 hours after initiation of the culturing process (e.g., the day the cells are seeded in the bioreactor to begin the culturing process).

In some embodiments, the initial temperature at the initiation of the culturing process is about 37° C. In certain embodiments, a first temperature shift comprises a temperature decrease from about 37° C. to about 30° C., from about 37° C. to about 31° C., from about 37° C. to about 32° C., from about 37° C. to about 33° C., from about 37° C. to about 34° C., or from about 37° C. to about 35° C. In certain embodiments, a second temperature shift comprises a temperature decrease from about 35° C. to about 30° C., from about 35° C. to about 31° C., from about 35° C. to about 32° C., from about 35° C. to about 33° C., from about 35° C. to about 34° C., about 34° C. to about 30° C., from about 34° C. to about 31° C., from about 34° C. to about 32° C., from about 34° C. to about 33° C., about 33° C. to about 30° C., from about 33° C. to about 31° C., or from about 33° C. to about 32° C.

In some embodiments, at least one, at least two, at least three, at least four or at least five pH shifts occur during the culturing process. In certain embodiments, a pH shift occurs at least one day, at least 2 days, at least 3 days, at least 4 days, or at least 5 days after initiation of the culturing process (e.g., the day the cells are seeded in the bioreactor to begin the culturing process), wherein said pH is set at about 6.65, about 6.70, about 6.75, about 6.80, about 6.85, about 6.90, or about 6.95. In certain embodiments, the pH is forced to a setpoint at least 3 days after initiation of the culturing process.

Culture Initiation

Typically, a desired cell expressing an AP-agent as described herein is first propagated in an initial culture by any of the variety of methods well-known to one of ordinary skill in the art. In some embodiments of the present invention, culture initiation occurs on the day the cells are seeded in the bioreactor to begin the culturing process. In some embodiments, the method of production according to the present invention include providing a seeding density of at least 0.4×10⁶ cells/mL, or at least 0.45×10⁶ cells/mL, or at least 0.5×10⁶ cells/mL, or at least 0.55×10⁶ cells/mL, or at least 0.6×10⁶ cells/mL, or at least 0.65×10⁶ cells/mL, or at least 0.7×10⁶ cells/mL, or at least 0.75×10⁶ cells/mL, or at least 0.8×10⁶ cells/mL, or at least 0.85×10⁶ cells/mL, or at least 0.90×10⁶ cells/mL, or at least 0.95×10⁶ cells/mL, or at least 1.0×10⁶cells/mL, or at least 1.05×10⁶ cells/mL, or at least 1.1×10⁶cells/mL, or at least 1.15×10⁶ cells/mL, or at least 1.2×10⁶ cells/mL, or at least 1.25×10⁶ cells/mL, or at least 1.3×10⁶ cells/mL, or at least 1.35×10⁶ cells/mL, or at least 1.4×10⁶ cells/mL, 1.45×10⁶ cells/mL, 1.5×10⁶ cells/mL, or at least 1.55×10⁶ cells/mL, or at least 1.6×10⁶ cells/mL, or at least 1.65×10⁶ cells/mL, or at least 1.7×10⁶ cells/mL, or at least 1.75×10⁶ cells/mL, or at least 1.8×10⁶ cells/mL, or at least 1.85×10⁶ cells/mL, or at least 1.90×10⁶ cells/mL, or at least 1.95×10⁶ cells/mL, or at least 2.0×10⁶ cells/mL.

Growth Phase

Typically, once the production bioreactor has been seeded as described above, the cell culture is maintained in the initial growth phase under conditions conducive to the survival, growth and viability of the cell culture. In accordance with the present invention, the production bioreactor can be any volume that is appropriate for production of proteins.

Transition Phase

In some embodiments, when the cells are ready for the production phase, the culture conditions may be changed to maximize the production of the protein of interest. Such culture condition changes typically take place in a transition phase. In some embodiments, such changes may include a shift in one or more of a number of culture conditions including, but not limited to, temperature, pH, osmolarity and culture medium.

Production Phase

In accordance with the present invention, once the cell culture reaches a desired cell density and viability, with or without a transition phase, the cell culture is maintained for a subsequent production phase under culture conditions conducive to the survival and viability of the cell culture and appropriate for expression of AP agent at adequate levels. In some embodiments, changes in one or more of a number of culture conditions can occur during the production phase.

Separating AP Producing Cells and Recovering AP-Based Agent

According to the present invention, the method includes a step of separating the AP-producing cells from the produced AP-based agent at a discrete time point over the course of the manufacturing culturing process. In some embodiments, the cells are separated by at least 10 days, at least 11 days, at least 12 days, at least 13 days, at least 14 days, at least 15 days, at least 16 days, at least 17 days, at least 18 days, at least 19 days, or at least 20 days after initiation of the culturing process.

According to the present invention, the method further includes a step of recovering the produced AP-based agent from the separated cells. In some embodiments, the AP-based agent is secreted from the AP-producing cell into the cell culture medium.

Purification of Expressed AP-Based Agent

Various methods may be used to purify or isolate AP-based agents produced according to various methods described herein. In some embodiments, the expressed AP-based agent is secreted into the medium and thus cells and other solids may be removed, as by centrifugation or filtering for example, as a first step in the purification process. Alternatively, or additionally, the expressed AP-based agent is bound to the surface of the host cell. In this embodiment, the host cells expressing the polypeptide or protein are lysed for purification. Lysis of host cells can be achieved by any number of means well known to those of ordinary skill in the art, including physical disruption by glass beads and exposure to high pH conditions.

The AP-based agent may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference). Protease inhibitors such as phenyl methyl sulfonyl fluoride (PMSF), leupeptin, pepstatin or aprotinin may be added at any or all stages of the purification process in order to reduce or eliminate degradation of the polypeptide or protein. Protease inhibitors are particularly desired when cells must be lysed in order to isolate and purify the expressed polypeptide or protein.

The expressed IAP can be measured by various product quality measurements, as known by one skilled in the art. For example, assays such as SEC-HPL (to yield percent dimer, percent monomer, percent total aggregates, and percent total fragments), protein content (concentration in g/L), enzyme activity, RP-HPLC purity (percent main peak), non-reduced CE-SDS (percent main peak), and residual CHO host protein ELISA (CHO HCP (ppm)) can be employed.

In some embodiments, the IAP comprises at least 80%, at least 81%, at least 82%, at least 83%, at least 84%, at least 85%, at least 86%, at least 87%, at least 88%, or at least 89%, at least 90%, at least 91%, at least 92%, at least 93%, at least 94%, at least 95%, at least 96%, at least 97%, at least 98%, or at least 99% purity as measured by RP-HPLC.

Bioreactors

In some embodiments, the culturing process occurs in a bioreactor. Bioreactors may be perfusion, batch, fed-batch, repeated batch, or continuous (e.g. a continuous stirred-tank reactor models), for example. A bioreactor can be of any size so long as it is useful for the culturing of mammalian cells. Typically, a bioreactor will be at least 1 liter and may be 10, 100, 250, 500, 1000, 2500, 5000, 8000, 10,000, 12,0000 liters or more, or any volume in between. In certain embodiments according to the present invention, the bioreactor is at least 1 L, at least 1.5 L, at least 2 L, at least 2.5 L, at least 3 L, at least 3.5 L, at least 4 L, at least 4.5 L, at least 5 L, at least 6 L, at least 7 L, at least 8 L, at least 9 L, at least 10 L, at least 15 L, at least 20 L, at least 25 L, at least 30 L, at least 35 L, at least 40 L, at least 45 L, at least 50 L, at least 55 L, at least 60 L, at least 65 L, at least 70 L, at least 75 L, at least 80 L, at least 85 L, at least 90 L, at least 95 L, at least 100 L, at least 125 L, at least 150 L, at least 175 L, at least 200 L, at least 225 L, at least 250 L, at least 275 L, at least 300 L, at least 350 L, at least 400 L, at least 450 L, or at least 500 L. In certain embodiments according to the present invention, the scale of the bioreactor is at least 3 L, 50 L, or 200 L. In some embodiments, the bioreactor comprises a size of 3 L, 50 L, or 200 L.

Monitoring Culture Conditions

In certain embodiments of the present invention, the practitioner may find it beneficial or necessary to periodically monitor particular conditions of the growing cell culture. Monitoring cell culture conditions allows the practitioner to determine whether the cell culture is producing enzyme agent at suboptimal levels or whether the culture is about to enter into a suboptimal production phase. In order to monitor certain cell culture conditions, it may be necessary to remove small aliquots of the culture for analysis.

As non-limiting examples, it may be beneficial or necessary to monitor temperature, pH, cell density, cell viability, integrated viable cell density, percent of dimers, the presence of metabolites (e.g., glutamine, insulin, lactate, NH₄⁺, Na⁺, K⁺), osmolality, osmolarity, or titer of the expressed AP-based agent. Numerous techniques are well known in the art that will allow one of ordinary skill in the art to measure these conditions. For example, cell density may be measured using a hemacytometer, a Coulter counter, or Cell density examination (CEDEX). Viable cell density may be determined by staining a culture sample with Trypan blue. Since only dead cells take up the Tryptan blue, viable cell density can be determined by counting the total number of cells, dividing the number of cells that take up the dye by the total number of cells, and taking the reciprocal. Alternatively, the level of the expressed AP-based agent can be determined by standard molecular biology techniques such as Coomassie staining of SDS-PAGE gels, Western blotting, Bradford assays, Lowry assays, Biuret assays, and UV absorbance or activity assay. Metabolites may be measured by a cell culture analyzer, and osmolality may be measured with an osmometer, both by methods known to those skilled in the art.

In some embodiments, it may be beneficial or necessary to monitor the amount of total active AP units at any given time during the culturing process. In some embodiments, the total active AP units are measured after the AP-based agents has been recovered. In some embodiments, the produced AP-based agent has total active AP units of at least 2.00×10⁶, at least 2.05×10⁶, at least 2.10×10⁶, at least 2.15×10⁶, at least 2.20×10⁶, at least 2.25×10⁶, at least 2.30×10⁶, at least 2.35×10⁶, at least 2.40×10⁶, at least 2.45×10⁶, at least 2.50×10⁶, 2.60×10⁶, at least 2.65×10⁶, at least 2.70×10⁶, at least 2.75×10⁶, at least 2.80×10⁶, at least 2.85×10⁶, at least 2.90×10⁶, or at least 2.95×10⁶.

The amount of dimers present in a titer can be determined using SEC-HPLC columns. A first peak of the chromatogram may consist of dimers, while the second peak is considered a monomer. The sum of both peaks will establish the 100% value of the calculated titer. Without wishing to be bound by theory, it is believed that increased dimerization yields improvements in a variety of endpoint measurements associated with alkaline phosphatase activity (e.g., specific activity and/or total activity).

Alkaline Phosphatase Endpoint Measurements

In some embodiments, it can be helpful or beneficial to measure AP activity after the AP-based agent has been recovered from the culturing process. In order to test alkaline phosphatase enzyme activity, assays known to those skilled in the art can be performed. For example, an endpoint AP activity assay and/or a kinetic AP activity assay can be used. An assay for specific activity can also be used and these assays are well-known to those skilled in the art.

In various embodiments, the AP-based agent of the invention possesses desirable characteristics, including, for example, high specific activity (expressed as U/mg). The specific activity can be calculated from the respective enzymatic activities divided by the concentrations derived from HPLC quantitation. In some embodiments, the activity and/or specific activity of the alkaline phosphatase-based agent is increased by at least 30%, or at least 35%, or at least 40%, or at least 45%, or at least 50% in the presence of a supplementary addition of ZnSO₄ as compared to absence of a supplemental addition of ZnSO₄ during the culturing process.

IAP Activity Assay

An endpoint AP activity assay utilizes purified alkaline phosphatase as a standard by which the activity of samples assayed are quantified. AP solution can also be used as an indicative control. Samples are tested using 2 replicate wells from which S.D. values are generated. Briefly, various samples are dissolved in Sodium dihydrogen phosphate buffer (NaH₂PO₄ 50mM+ZnSO₄ 0.5 mM, pH 7.0). A standard curve of AP concentrations of the Sigma standard ranging from 0-20 nM is prepared alongside the AP samples. 80 μl of samples or standards are added to the wells of a flat bottomed 96-well plate, followed by 50 μl of 5 mM pNPP solution. The plate is then incubated for one hour at 25° C. in a light protected environment. After one hour, 20 μl of stop solution is added to each well, then the OD at A₄₀₅ is read in a plate reader, and concentrations are derived through comparison to the standard curve generated through a linear fit trend line, the Y=X equation of which is used to calculate concentration values.

In various embodiments, the AP-based agent of the invention possesses desirable characteristics, including, for example, high total AP activity (U/mL).

In various embodiments, the produced AP-based agent of the present invention possesses a total alkaline phosphatase activity of at least about 100 U/mL to about 5,000 U/mL. In various embodiments, the produced AP-based agent of the invention possesses a total AP activity of at least about 100 U/mL, about 200 U/mL, about 300 U/mL, about 400 U/mL, about 500 U/mL, about 600 U/mL, about 610 U/mL, about 620 U/mL, about 630 U/mL, about 640 U/mL, about 650 U/mL, about 660 U/mL, about 670 U/mL, about 680 U/mL, about 690 U/mL, about 700 U/mL, about 800 U/mL, about 900 U/mL, about 1,000 U/mL, about 1,100 U/mL, about 1,200 U/mL, about 1,300 U/mL, about 1,400 U/mL, about 1,500 U/mL, about 1,600 U/mL, about 1,700 U/mL, about 1,800 U/mL, about 1,900 U/mL, about 2,000 U/mL, about 3,000 U/mL, about 4,000 U/mL, or about 5,000 U/mL. In specific embodiments, the total AP activity is about 1,990 U/mL.

Kinetic IAP Activity Assay

A kinetic AP activity assay utilizes purified alkaline phosphatase as a control to test the activity of samples assayed. AP solution can also be used as an indicative control. Briefly, various samples are dissolved in diethanolamine based buffer (pH 9.8 at 37° C.), and after five minutes of pre-incubation at 37° C., are combined with a 5 mM solution of p-nitrophenyl phosphate (pNPP). After an additional 10 minutes, the colorimetric output at 405 nm as a function of pNPP→NPP dephosporylation via enzyme phosphatase activity is measured every 20 seconds over 5 minutes using a plate reader. This provides a readout of enzyme kinetics over this time period, the slope of which can be converted to enzyme activity using the substrate extinction coefficient (18.5 OD₄₀₅ units/mM*cm pathlength) or which can be compared to the slope generated from the AP standard.

In various embodiments, the alkaline phosphatase of the present invention possesses a specific activity of at least about 100 U/mg to about 20,000 U/mg. In various embodiments, the alkaline phosphatase of the invention possesses a specific activity of at least about 100 U/mg, about 200 U/mg, about 300 U/mg, about 400 U/mg, about 500 U/mg, about 600 U/mg, about 610 U/mg, about 620 U/mg, about 630 U/mg, about 640 U/mg, about 650 U/mg, about 660 U/mg, about 670 U/mg, about 680 U/mg, about 690 U/mg, about 700 U/mg, about 800 U/mg, about 900 U/mg, about 1,000 U/mg, about 1,100 U/mg, about 1,200 U/mg, about 1,300 U/mg, about 1,400 U/mg, about 1,500 U/mg, about 1,600 U/mg, about 1,700 U/mg, about 1,800 U/mg, about 1,900 U/mg, about 2,000 U/mg, about 3,000 U/mg, about 4,000 U/mg, about 5,000 U/mg, about 6,000 U/mg, about 7,000 U/mg, about 8,000 U/mg, about 9,000 U/mg, about 10,000 U/mg, about 11,000 U/mg, about 12,000 U/mg, about 13,000 U/mg, about 14,000 U/mg, about 15,000 U/mg, about 16,000 U/mg, about 17,000 U/mg, about 18,000 U/mg, about 19,000 U/mg, or about 20,000 U/mg. In specific embodiments, the specific activity is about 615 U/mg. In certain embodiments, the specific activity is at least about 1,200 U/mg.

### Pharmaceutical Composition and Administration

The present invention is directed, in part, to pharmaceutical compositions, formulations, and uses of one or more alkaline phosphatase-based agents (AP-based agents). Alkaline phosphatases are dimeric metalloenzymes that catalyze the hydrolysis of phosphate esters and dephosphorylate a variety of target substrates at physiological and higher pHs.

Formulations

The present invention provides the described AP-based agent and/or pharmaceutical compositions (and/or additional therapeutic agents) in various formulations. Any AP-based agent and/or pharmaceutical composition (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the AP-based agent and/or pharmaceutical compositions (and/or additional therapeutic agents) may conveniently be presented in unit dosage forms. For example, the dosage forms may be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting)

In one embodiment, the AP-based agent (and/or additional therapeutic agents) described herein is formulated as a composition adapted for a mode of administration described herein.

In various embodiments, the administration the AP-based agent and/or pharmaceutical compositions (and/or additional therapeutic agents) is any one of oral, intravenous, and parenteral. For example, routes of administration include, but are not limited to, oral, intradermal, intramuscular, intraperitoneal, intravenous, subcutaneous, intranasal, epidural, sublingual, intranasal, intracerebral, intravaginal, transdermal, rectally, by inhalation, or topically (e.g., to the ears, nose, eyes, or skin).

In one embodiment, the AP-based agent and/or pharmaceutical compositions (and/or additional therapeutic agents) described herein is formulated as a composition adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide a sustained action over an extended period of time. Selectively permeable membranes surrounding an osmotically active agent driving any alkaline phosphatase (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, ethacrylic acid and derivative polymers thereof, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various embodiments, the AP-based agent and/or pharmaceutical compositions (and/or additional therapeutic agent) are formulated as solid dosage forms such as tablets, dispersible powders, granules, and capsules. In one embodiment, the AP-based agent and/or pharmaceutical compositions (and/or additional therapeutic agent) are formulated as a capsule. In another embodiment, the AP-based agent and/or pharmaceutical compositions (and/or additional therapeutic agent) are formulated as a tablet. In yet another embodiment, the AP-based agent and/or pharmaceutical compositions (and/or additional therapeutic agent) are formulated as a soft-gel capsule. In a further embodiment, the AP-based agent and/or pharmaceutical compositions (and/or additional therapeutic agent) are formulated as a gelatin capsule.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents.

In various embodiments, the formulations of the AP-based agents may additionally comprise a pharmaceutically acceptable carrier or excipient. As one skilled in the art will recognize, the formulations can be in any suitable form appropriate for the desired use and route of administration.

In some dosage forms, the agents described herein are mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients may have two or more functions in the oral dosage form. In the case of an oral dosage form, for example, a capsule or a tablet, the dosage form may also comprise buffering agents.

The formulation can additionally include a surface active agent. Surface active agents suitable for use in the present invention include, but are not limited to, any pharmaceutically acceptable, non-toxic surfactant. Classes of surfactants suitable for use in the compositions of the invention include, but are not limited to polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some embodiments, compositions of the invention may comprise one or more surfactants including, but not limited to, sodium lauryl sulfate, polysorbate 20, polysorbate 40, polysorbate 60, polysorbate 80, and triethyl citrate.

The formulation can also contain pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

The formulation can also include one or more application solvents. Some of the more common solvents that can be used to apply, for example, a delayed-release coating composition include isopropyl alcohol, acetone, methylene chloride and the like.

The formulation can also include one or more alkaline materials. Alkaline material suitable for use in compositions of the invention include, but are not limited to, sodium, potassium, calcium, magnesium and aluminum salts of acids such as phosphoric acid, carbonic acid, citric acid and other aluminum/magnesium compounds. In addition the alkaline material may be selected from antacid materials such as aluminum hydroxides, calcium hydroxides, magnesium hydroxides and magnesium oxide.

In various embodiments, the formulation can additionally include magnesium and/or zinc. Without wishing to be bound by theory, the inclusion of magnesium and/or zinc in the formulation promotes protein folding (e.g., dimer formation) and bioactivity of the AP-based agent. In some embodiments, the formulation can include magnesium at a concentration of from about 1 μM to greater than 5 mM (e.g., from about 1 μM to more than 5 mM), inclusive of all ranges and values therebetween. In some embodiments, the formulation can include zinc at a concentration of about 1 μM to greater than 1 mM (e.g., from about 1 μM to more than 1 mM), inclusive of all ranges and values therebetween. In various embodiments, the formulation of the present invention is substantially free of metal chelators.

In various embodiments, the pH of the formulation ensures that the AP-based agent is properly folded (e.g., dimer formation) and is bioactive. In some embodiments, the formulation is maintained at a pH such that the amino acids which coordinate the binding of magensium and/or zinc within the AP-based agent are not protonated. Protonation of such coordinating amino acids may lead to loss of metal ions and bioactivity and dimer disassociation. In various embodiments, the pH of the formulation is greater than about 6, about 6.5, about 7, about 7.5, about 8, about 8.5, about 9, about 9.5, about 10, about 10.5, about 11, about 11.5, or about 12.

Besides inert diluents, the oral compositions can also include adjuvants such as sweetening, flavoring, and perfuming agents.

In various embodiments, the AP-based agent and/or pharmaceutical compositions (and/or additional therapeutic agents) are formulated for systemic or local delivery. In an embodiment, administration is systemic. In another embodiment, it may be desirable to administer locally to the area in need of treatment.

Various methods may be used to formulate and/or deliver the agents described herein to a location of interest. For example, the alkaline phosphatase and/or pharmaceutical compositions (and/or additional therapeutic agents) described herein may be formulated for delivery to the gastrointestinal tract. The gastrointestinal tract includes organs of the digestive system such as mouth, esophagus, stomach, duodenum, small intestine, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the alkaline phosphatases and/or pharmaceutical compositions (and/or additional therapeutic agents) described herein may be formulated for delivery to one or more of the stomach, small intestine, large intestine and rectum and includes all subsections thereof (e.g. duodenum, jejunum and ileum, colon transversum, colon descendens, colon ascendens, colon sigmoidenum and cecum). In some embodiments, the compositions described herein may be formulated to deliver to the upper or lower GI tract. In an embodiment, the alkaline phosphatases and/or pharmaceutical compositions (and/or additional therapeutic agents) may be administered to a subject, by, for example, directly or indirectly contacting the mucosal tissues of the gastrointestinal tract.

In various embodiments, the administration the AP-based agent and/or pharmaceutical compositions (and/or additional therapeutic agents) is into the GI tract via, for example, oral delivery, nasogastral tube, intestinal intubation (e.g. an enteral tube or feeding tube such as, for example, a jejunal tube or gastro jejunal tube, etc.), direct infusion (e.g., duodenal infusion), endoscopy, colonoscopy, or enema.

For example, in various embodiments, the present invention provides modified release formulations comprising at least one AP-based agent (and/or additional therapeutic agents), wherein the formulation releases a substantial amount of the AP-based agent (and/or additional therapeutic agents) into one or more regions of the GI tract. For example, the formulation may release at least about 60% of the AP-based agent after the stomach and into one or more regions of the GI tract.

In various embodiments, the modified-release formulation of the present invention releases at least 60% of the AP-based agent (or additional therapeutic agents) after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the AP-based agent (or additional therapeutic agents) in the intestines.

In various embodiments, the modified-release formulation of the present invention releases at least 60% of the AP-based agent (or additional therapeutic agents) in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the AP-based agent (or additional therapeutic agents) in the small intestine (e.g., one or more of duodenum, jejunum, ileum, and ileocecal junction).

In various embodiments, the modified-release formulation of the present invention releases at least 60% of the AP-based agent (or additional therapeutic agents) in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the AP-based agent (or additional therapeutic agents) in the large intestine (e.g., one or more of cecum, ascending, transverse, descending or sigmoid portions of the colon, and rectum).

In various embodiments, the modified-release formulation does not substantially release the AP-based agent (or additional therapeutic agents) in the stomach.

In certain embodiments, the modified-release formulation releases the AP-based agent (or additional therapeutic agents) at a specific pH. For example, in some embodiments, the modified-release formulation is substantially stable in an acidic environment and substantially unstable (e.g., dissolves rapidly or is physically unstable) in a near neutral to alkaline environment. In some embodiments, stability is indicative of not substantially releasing while instability is indicative of substantially releasing. For example, in some embodiments, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some embodiments, the present formulations are stable in lower pH areas and therefore do not substantially release in, for example, the stomach. In some embodiments, modified-release formulation is substantially stable at a pH of about 1 to about 4 or lower and substantially unstable at pH values that are greater. In these embodiments, the modified-release formulation does not substantially release in the stomach. In these embodiments, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In some embodiments, modified-release formulation is substantially stable at a pH of about 4 to about 5 or lower and consequentially is substantially unstable at pH values that are greater and therefore is not substantially released in the stomach and/or small intestine (e.g. one or more of the duodenum, jejunum, and ileum). In these embodiments, the modified-release formulation substantially releases in the large intestine (e.g. one or more of the cecum, ascending colon, transverse colon, descending colon, and sigmoid colon). In various embodiments, the pH values recited herein may be adjusted as known in the art to account for the state of the subject, e.g. whether in a fasting or postprandial state.

In some embodiments, the modified-release formulation is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In some embodiments, the modified-release formulation is stable in gastric fluid or stable in acidic environments. These modified-release formulations release about 30% or less by weight of the alkaline phosphatase and/or additional therapeutic agent in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of the of the invention may release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the alkaline phosphatase and/or additional therapeutic agent in the modified-release formulation in gastric fluid with a pH of 4-5, or less or simulated gastric fluid with a pH of 4-5 or less, in about 15, or about 30, or about 45, or about 60, or about 90 minutes. Modified-release formulations of the invention may release about 1%, about 2%, about 3%, about 4%, about 5%, about 6%, about 7%, about 8%, about 9%, or about 10% by weight of the total alkaline phosphatase and/or additional therapeutic agent in the modified-release formulation in gastric fluid with a pH of 5 or less, or simulated gastric fluid with a pH of 5 or less, in about 15, or about 30, or about 45, or about 60, or about 90 minutes.

In some embodiments, the modified-release formulation is unstable in intestinal fluid. These modified-release formulations release about 70% or more by weight of the alkaline phosphatase and/or additional therapeutic agent in the modified-release formulation in intestinal fluid or simulated intestinal fluid in about 15, or about 30, or about 45, or about 60, or about 90 minutes. In some embodiments, the modified-release formulation is unstable in near neutral to alkaline environments. These modified-release formulations release about 70% or more by weight of the alkaline phosphatase and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments may release 70% or more by weight of alkaline phosphatase and/or additional therapeutic agent in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various embodiments, the modified-release formulation of the invention is substantially stable in chyme. For example, there is, in some embodiments, a loss of less than about 50% or about 40%, or about 30%, or about 20%, or about 10% of AP-based agent activity in about 10, or 9, or 8, or 7, or 6, or 5, or 4, or 3, or 2, or 1 hour from administration.

In various embodiments, the modified-release formulations of the present invention are designed for immediate release (e.g. upon ingestion). In various embodiments, the modified-release formulations may have sustained-release profiles, i.e. slow release of the active ingredient(s) in the body (e.g., GI tract) over an extended period of time. In various embodiments, the modified-release formulations may have a delayed-release profile, i.e. not immediately release the active ingredient(s) upon ingestion; rather, postponement of the release of the active ingredient(s) until the composition is lower in the gastrointestinal tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various embodiments, the modified-release formulation of the present invention may utilize one or more modified-release coatings such as delayed-release coatings to provide for effective, delayed yet substantial delivery of the alkaline phosphatase to the GI tract together with, optionally, additional therapeutic agents.

In various embodiments, the modified-release formulation of the present invention may utilize one or more modified-release coatings such as delayed-release coatings to provide for effective, delayed yet substantial delivery of the alkaline phosphatase to the intestines together with, optionally, other additional therapeutic agents.

In one embodiment, the delayed-release coating includes an enteric agent that is substantially stable in acidic environments and substantially unstable in near neutral to alkaline environments. In an embodiment, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and Eudragit®-type polymer (poly(methacrylic acid, methylmethacrylate), hydroxypropyl methylcellulose acetate succinate, cellulose acetate trimellitate, shellac or other suitable enteric coating polymers. The Eudragit®-type polymers include, for example, Eudragit® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. Similar polymers include Kollicoat® MAE 30 DP and Kollicoat® MAE 100 P. In some embodiments, one or more of Eudragit® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In various embodiments, the enteric agent may be a combination of the foregoing solutions or dispersions. In an embodiment, the delayed-release coating includes the enteric agent Eudragit® L 30 D-55.

In certain embodiments, one or more coating system additives are used with the enteric agent. For example, one or more PlasACRYL™ additives may be used as an anti-tacking agent coating additive. Illustrative PlasACRYL™ additives include, but are not limited to PlasACRYL™ HTP20 and PlasACRYL™ T20. In an embodiment, PlasACRYL™ HTP20 is formulated with Eudragit® L 30 D-55 coatings. In another embodiment, PlasACRYL™ T20 is formulated with Eudragit® FS 30 D coatings.

In another embodiment, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include Eudragit RS®, Eudragit RL®, and Eudragit NE®. Insoluble polymers useful in the present invention include polyvinyl esters, polyvinyl acetals, polyacrylic acid esters, butadiene styrene copolymers, and the like. In one embodiment, colonic delivery is achieved by use of a slowly-eroding wax plug (e.g., various PEGs, including for example, PEG6000).

In a further embodiment, the delayed-release coating may be degraded by a microbial enzyme present in the gut flora. In one embodiment, the delayed-release coating may be degraded by a bacteria present in the small intestine. In another embodiment, the delayed-release coating may be degraded by a bacteria present in the large intestine.

In various embodiments, the modified release formulation is designed for release in the colon. Various colon-specific delivery approaches may be utilized. For example, the modified release formulation may be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation may be designed as a core tablet with three layers of polymer. The first coating is an acid-soluble polymer (e.g., Eudragit E®), the outer coating is enteric, along with a hydroxypropyl methylcellulose barrier layer interposed in between. In another embodiment, colon delivery may be achieved by formulating the alkaline phosphatase (and/or additional therapeutic agent) with specific polymers that degrade in the colon such as, for example, pectin. The pectin may be further gelled or crosslinked with a cation such as a zinc cation. In an embodiment, the formulation is in the form of ionically crosslinked pectin beads which are further coated with a polymer (e.g., Eudragit® polymer). Additional colon specific formulations include, but are not limited to, pressure-controlled drug delivery systems (prepared with, for example, ethylcellulose) and osmotic controlled drug delivery systems (i.e., ORDS-CT).

Formulations for colon specific delivery of the AP-based agent (and/or additional therapeutic agents), as described herein, may be evaluated using, for example, in vitro dissolution tests. For example, parallel dissolution studies in different buffers may be undertaken to characterize the behavior of the formulations at different pH levels. Alternatively, in vitro enzymatic tests may be carried out. For example, the formulations may be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further embodiment, in vivo evaluations may be carried out using animal models such as dogs, guinea pigs, rats, and pigs. Further, clinical evaluation of colon specific drug delivery formulations may be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon may be monitored by colonoscopy and intubation.

In various embodiments, the present formulation provides for substantial uniform dissolution of the AP-based agent (and/or additional therapeutic agent) in the desired area of release in the GI tract. In an embodiment, the present formulation minimizes patchy or heterogeneous release of the AP-based agent.

In various embodiments, the present invention provides for modified-release formulations that release multiple doses of the AP-based agent, at different locations along the intestines, at different times, and/or at different pH. In an illustrative embodiment, the modified-release formulation comprises a first dose of the AP-based agent and a second dose of the AP-based agent, wherein the first dose and the second dose are released at different locations along the intestines, at different times, and/or at different pH. For example, the first dose is released at the duodenum, and the second dose is released at the ileum. In another example, the first dose is released at the jejunum, and the second dose is released at the ileum. In other embodiments, the first dose is released at a location along the small intestine (e.g., the duodenum), while the second dose is released along the large intestine (e.g., the ascending colon). In various embodiments, the modified-release formulation may release at least one dose, at least two doses, at least three doses, at least four doses, at least five doses, at least six doses, at least seven doses, or at least eight doses of the AP-based agent at different locations along the intestines, at different times, and/or at different pH. Further the dual pulse description herein applies to modified-release formulations that release the AP-based agent and an additional therapeutic agent.

In various embodiments, the invention provides a formulation comprising: a core particle having a base coat comprising one or more AP-based agents, and a delayed-release coating disposed over the coated core particle. The delayed-release coating may be substantially stable in acidic environments and/or gastric fluid, and/or substantially unstable in near neutral to alkaline environments or intestinal fluid thereby exposing the coated core particle to intestinal fluid. The base coat comprising one or more AP-based agents may further comprise one or more additional therapeutic agents. Optionally a plurality of base coats may be applied to the core particle each of which may contain an AP-based agent and/or an additional therapeutic agent. In an embodiment, the core particle includes sucrose. In an embodiment, an AP-based agent can be sprayed onto an inert core (e.g., a sucrose core) and spray-dried with an enteric layer (e.g., Eudragit® L30 D-55) to form pellets or beads containing AP-based agents.

Optionally, the core particle may comprise one or more AP-based agents and/or one or more additional therapeutic agents. In one embodiment, one or more doses of the AP-based agent may be encapsulated in a core particle, for example, in the form of a microsphere or a mini-sphere. For example, the AP-based agent may be combined with a polymer (e.g., latex), and then formed into a particulate, micro-encapsulated enzyme preparation, without using a sucrose core. The microspheres or mini-spheres thus formed may be optionally covered with a delayed-release coating.

A variety of approaches for generating particulates (such as microspheres, mini-spheres, aggregates, other) may be utilized for the inclusion of enzymatic proteins. They typically involve at least two phases, one containing the protein, and one containing a polymer that forms the backbone of the particulate. Most common are coacervation, where the polymer is made to separate from its solvent phase by addition of a third component, or multiple phase emulsions, such as water in oil in water (w/o/w) emulsion where the inner water phase contains the protein, the intermediate organic phase contains the polymer, and the external water phase stabilizers that support the w/o/w double emulsion until the solvents can be removed to form, for example, microspheres or mini-spheres. Alternatively, the alkaline phosphatase and stabilizing excipients (for example, trehalose, mannitol, Tween 80, polyvinyl alcohol) are combined and sprayed from aqueous solution and collected. The particles are then suspended in a dry, water immiscible organic solvent containing polymer and release modifying compounds, and the suspension sonicated to disperse the particles. An additional approach uses aqueous phases but no organic solvent. Specifically, the enzymatic protein, buffer components, a polymer latex, and stabilizing and release-modifying excipients are dissolved/dispersed in water. The aqueous dispersion is spray-dried, leading to coalescence of the latex, and incorporation of the protein and excipients in particles of the coalesced latex. When the release modifiers are insoluble at acidic conditions but soluble at higher pHs (such as carboxylic acid) then release from the matrix is inhibited in the gastric environment. In an embodiment, alkaline phosphatase may be initially solubilized as an emulsion, microemulsion, or suspension and then formulated into solid mini-spheres or microspheres. The formulation may then be coated with, for example, a delayed-release, sustained-release, or controlled-release coating to achieve delivery at a specific location such as, for example, the intestines.

In various embodiments, the formulation may comprise a plurality of modified-release particles or beads or pellets or microspheres. In an embodiment, the formulation is in the form of capsules comprising multiple beads. In another embodiment, the formulation is in the form of capsules comprising multiple pellets. In another embodiment, the formulation is in the form of capsules comprising multiple microspheres or mini-spheres.

In some embodiments, before applying the delayed-release coating to the coated core particle, the particle can optionally be covered with one or more separating layers comprising pharmaceutical excipients including alkaline compounds such as for instance pH-buffering compounds. The separating layer essentially separates the coated core particle from the delayed-release coating.

The separating layer can be applied to the coated core particle by coating or layering procedures typically used with coating equipment such as a coating pan, coating granulator or in a fluidized bed apparatus using water and/or organic solvents for the coating process. As an alternative the separating layer can be applied to the core material by using a powder coating technique. The materials for separating layers are pharmaceutically acceptable compounds such as, for instance, sugar, polyethylene glycol, polyvinylpyrrolidone, polyvinyl alcohol, polyvinyl acetate, hydroxypropyl cellulose, methyl-cellulose, ethylcellulose, hydroxypropyl methylcellulose, carboxymethylcellulose sodium and others, used alone or in mixtures. Additives such as plasticizers, colorants, pigments, fillers, anti-tacking and anti-static agents, such as for instance magnesium stearate, sodium stearyl fumarate, titanium dioxide, talc and other additives can also be included in the separating layer.

In some embodiments, the coated particles with the delayed-release coating may be further covered with an overcoat layer. The overcoat layer can be applied as described for the other coating compositions. The overcoat materials are pharmaceutically acceptable compounds such as sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. The overcoat materials can prevent potential agglomeration of particles coated with the delayed-release coating, protect the delayed-release coating from cracking during the compaction process or enhance the tableting process.

In various embodiments, the formulations of the present invention take the form of those as described in International Patent Application No. PCT/US15/54606 and those as described in U.S. Patent Publication 2017/0009217 A1, the entire contents of all of which are incorporated herein by reference.

In various embodiments, the formulations of the present invention take the form of those as described in one or more of U.S. Pat. Nos. 8,535,713 and 8,9117,77 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various embodiments, the formulations of the present invention take the form of those as described in International Patent Publication No. WO 2008/135090, the contents of which are hereby incorporated by reference in their entirety.

In various embodiments, the formulations of the present invention take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

In various embodiments, the process of formulating the AP-based agent is sufficiently gentle such that the tertiary structure of the AP-based agent (e.g., dimeric structure) is substantially intact. In various embodiments, the process of formulating the AP-based agent includes a step of refolding the AP-based agent. In such embodiments, the step of refolding the AP-based agent may include the addition of magnesium and/or cyclodextrin.

Administration and Dosages

AP-based agents may be administered to patients suffering from GI complications etc. in accordance with known methods. For example, AP-based agents may be delivered intravenously, subcutaneously, intramuscularly, parenterally, transdermally, or transmucosally (e.g., orally or nasally).

It will be appreciated that the actual dose of the AP-based agent to be administered according to the present invention will vary according to the particular compound, the particular dosage form, and the mode of administration. Many factors that may modify the action of the AP-based agent (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

Individual doses of the AP-based agent can be administered in unit dosage forms (e.g., tablets or capsules) containing, for example, from about 0.01 mg to about 1,000 mg, about 0.01 mg to about 900 mg, about 0.01 mg to about 800 mg, about 0.01 mg to about 700 mg, about 0.01 mg to about 600 mg, about 0.01 mg to about 500 mg, about 0.01 mg to about 400 mg, about 0.01 mg to about 300 mg, about 0.01 mg to about 200 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, or from about 0.1 mg to about 1 mg active ingredient per unit dosage for. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg of the AP-based agent, inclusive of all values and ranges therebetween.

In one embodiment, the AP-based agent is administered at an amount of from about 0.01 mg to about 1,000 mg daily, about 0.01 mg to about 900 mg daily, about 0.01 mg to about 800 mg daily, about 0.01 mg to about 700 mg daily, about 0.01 mg to about 600 mg daily, about 0.01 mg to about 500 mg daily, about 0.01 mg to about 400 mg daily, about 0.01 mg to about 300 mg daily, about 0.01 mg to about 200 mg daily, about 0.01 mg to about 100 mg daily, an amount of from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the AP-based agent is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg, inclusive of all values and ranges therebetween.

In some embodiments, a suitable dosage of the AP-based agent is in a range of about 0.01 mg/kg to about 100 mg/kg of body weight of the subject, about 0.01 mg/kg to about 90 mg/kg of body weight of the subject, about 0.01 mg/kg to about 80 mg/kg of body weight of the subject, about 0.01 mg/kg to about 70 mg/kg of body weight of the subject, about 0.01 mg/kg to about 60 mg/kg of body weight of the subject, about 0.01 mg/kg to about 50 mg/kg of body weight of the subject, about 0.01 mg/kg to about 40 mg/kg of body weight of the subject, about 0.01 mg/kg to about 30 mg/kg of body weight of the subject, about 0.01 mg/kg to about 20 mg/kg of body weight of the subject, about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other embodiments, a suitable dosage of the AP-based agent is in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain embodiments of the invention, the AP-based agent may be administered, for example, more than once daily (e.g., about two, about three, about four, about five, about six, about seven, about eight, about nine, or about ten times per day), about once per day, about every other day, about every third day, about once a week, about once every two weeks, about once every month, about once every two months, about once every three months, about once every six months, or about once every year.

### Methods of Treatment

Without wishing to be bound by theory, it is believed that AP-based agent including alkaline phosphatases (e.g., IAPs) play a key role in many GI and systemic processes including, for example, participating in intestinal defense, mediating anti-inflammatory functions, maintaining normal gut microflora profiles, maintaining mucosal barrier integrity, and regulating digestion and nutrient (fat) absorption. Accordingly, the present invention provides the use of AP-based agents in a broad-range of therapeutic applications for modulating immune functions, metabolic functions, and neurological functions. In various embodiments, the present invention provides for the treatment of microbiome-related disorders, GI dysbiosis, GI inflammation, colitis (e.g., ulcerative colitis, Crohn's disease, acute and chronic radiation enteropathy, colitis and proctitis), metabolic diseases (e.g., metabolic syndrome, obesity, cachexia, NASH and diabetes), neurological diseases (e.g., multiple sclerosis, neuropsychiatric disorders), cystic fibrosis, sepsis, acute kidney injury (AM) and renal failure with an AP, including, without limitation a pharmaceutical composition comprising an AP-based agent, such as the modified release formulations described herein.

In various aspects, the present invention provides methods for modulating and protecting a subject's GI microbiome, comprising administering an effective amount of a pharmaceutical composition comprising an AP-based agent (and/or additional therapeutic agents) to the subject. In some embodiments, methods of the invention may be used to treat subjects with reduced levels and/or function of GI tract microbiome by administering an AP-based agent of the invention so as to increase or preserve the number of commensal bacteria and composition of the GI microbiome. In other embodiments, methods of the invention relate to treating infections by pathogenic bacteria and/or inhibiting the growth or decrease the number of pathogenic bacteria in the GI tract.

In various embodiments, the methods of the invention comprise treating or preventing a microbiome-mediated disorder. Illustrative microbiome-mediated disorder includes, but are not limited to, for example, those found in Table 3 of WO 2014/121298, the entire contents of which are incorporated herein by reference. For example, the methods described can be used to treat symptoms associated with reduced levels of commensal bacteria and/or function of GI tract microbiome, e.g., antibiotic-associated diarrhea (AAD), Clostridioides difficile (formerly Clostridium difficile)-associated disease (CDAD), inflammatory disorders, acquired immunodeficiency syndrome (AIDS) including HIV-mediated gut dysbiosis and GI barrier dysfunctions, hypothyroidism, and obesity.

Treatment of CDI and/or CDAC

In various aspects, the present invention provides pharmaceutical compositions comprising an AP-based agent of the invention (and/or additional therapeutic agents) for use in treating an antibiotic-induced adverse effect in the GI tract and/or prevention or treatment of CDI and/or a CDAD in a subject in need thereof. Without wishing to be bound by theory, it is believed that AP-based agent of the invention mediates NTP dephosphorylation which promotes the growth of commensal bacteria in preference to pathologic bacteria and hasten the recovery from antibiotic-induced dysbiosis. Accordingly, treatment with the AP-based agents of the invention has the potential to protect from CDI and enteric gram negative pathogens. In various embodiments, the antibiotic-induced adverse effect and/or CDI or CDAD is one or more of: antibiotic-associated diarrhea, C. difficile diarrhea (CDD), C. difficile intestinal inflammatory disease, colitis, pseudomembranous colitis, fever, abdominal pain, dehydration and disturbances in electrolytes, megacolon, peritonitis, and perforation and/or rupture of the colon.

In various embodiments, the subjects include, but are not limited to, subjects that are at a particular risk for a microbiome-mediated disorder, such as, by way of non-limiting example, those undergoing treatment or having recently undergone treatment with an antibiotic. For example, the subject may have taken an antibiotic during the past about 30 or so days and/or have an immune system that is weak (e.g. from a chronic illness) and/or is a woman and/or is elderly (e.g. over about 65 years old) and/or is undergoing (or has undergone) treatment with for heartburn or stomach acid disorders (e.g. with agents such as PREVACID, TAGAMET, PRILOSEC, or NEXIUM and related drugs) and/or has recently been in the hospital, including in an intensive care unit, or lives in a nursing home. Accordingly, in some embodiments, the methods and uses of the present invention treat or prevent a nosocomial infection and/or a secondary emergent infection and/or a hospital acquired infection (HAI).

In various embodiments, the present invention provides methods for treating antibiotic-induced adverse effects in the GI tract, comprising administration of an effective amount of an alkaline phosphatase of the invention (and/or additional therapeutic agents) to a subject in need thereof. In another embodiment, the present invention provides methods for preventing an antibiotic-induced adverse effect in the GI tract, comprising an effective amount of an alkaline phosphatase of the invention (and/or additional therapeutic agents) to a subject in need thereof.

In various embodiments, the alkaline phosphatase of the invention protects the intestinal microbiome from antibiotics-induced damage. In an embodiment, the AP-based agent protects the intestinal microbiome from cephalosporin-induced damage. In some embodiments, the AP-based agent of the invention protects the intestinal microbiome from ceftriaxone (CRO)-induced damage. In some embodiments, the methods of the invention treat or prevent an antibiotics-associated adverse effect including but not limited to diarrhea, nausea, vomiting, dysgeusia, colitis, and pseudomembranous colitis disease and/or symptoms. In an embodiment, methods of the invention can be used to treat or prevent antibiotic-associated diarrhea (AAD).

In various embodiments, the present invention provides for compositions and methods for treating infections by pathogenic bacteria and/or inhibiting the growth or decrease the number of pathogenic bacteria in the GI tract. In various embodiments, the present invention provides for compositions and methods that mitigate or prevent the overgrowth of various coliforms in a patient's gut (including coliforms that are virulent and/or antibiotic resistant). Illustrative coliforms include Citrobacter, Enterobacer, Hafnia, Kelbsiella, and Escherichia. In various aspects, the methods and compositions described herein prevent or diminish secondary infections with resistant organisms. In an embodiment, the pathogenic bacteria is an enterobacteria such as Salmonella.

In various embodiments, the present invention provides methods for treating or preventing CDI and/or a CDAD, comprising administering an effective amount of an alkaline phosphatase of the invention a subject in need thereof. In an embodiment, the present invention provides methods for preventing CDI and/or a CDAD, comprising administering an effective amount of administering an effective amount of an alkaline phosphatase of the invention to a subject in need thereof (by way of non-limiting example, a patient that is being administered or will be administered an antibiotic).

In some embodiments, the invention relates to a method of preventing CDI and/or a CDAD, comprising administering an effective amount of an alkaline phosphatase of the invention to a subject in need thereof, wherein the subject is undergoing therapy with a primary antibiotic. A “primary antibiotic” refers to an antibiotic that is administered to a patient and which may result in CDI and/or CDAD. These include the antibiotics that most often lead to CDI and/or CDAD: e.g., fluoroquinolones, cephalosporins, clindamycin and penicillins. In some embodiments the antibiotic is a selected from beta-lactams, carbapenems, monobactams, β-lactamase inhibitors, aminoglycosides, tetracyclines, rifamycins, macrolides, ketolides, lincosamides, streptogramins, sulphonamides, oxazolidinones, and quinolones. In some embodiments the antibiotic includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, and methicillin); monobactam antibiotics (aztreonam); and carbapenem antibiotics (ertapenem, doripenem, imipenem/cilastatin, and meropenem), and vancomycin.

In various embodiments, the CDI and/or CDAD is treated or prevented in the context of initial onset or relapse/recurrence (e.g. due to continued or restarted antibiotic therapy). For example, in a patient that has previously suffered from CDI, the present alkaline phosphatase may be administered upon the first symptoms of recurrence. By way of non-limiting example, symptoms of recurrence include, in a mild case, about 5 to about 10 watery bowel movements per day, no significant fever, and only mild abdominal cramps while blood tests may show a mild rise in the white blood cell count up to about 15,000 (normal levels are up to about 10,000), and, in a severe case, more than about 10 watery stools per day, nausea, vomiting, high fever (e.g. about 102-104° F.), rectal bleeding, severe abdominal pain (e.g. with tenderness), abdominal distention, and a high white blood count (e.g. of about 15,000 to about 40,000).

Regardless of initial onset or relapse/recurrence, CDI and/or CDAD may be diagnosed via any of the symptoms described herein (e.g. watery diarrhea about 3 or more times a day for about 2 days or more, mild to bad cramping and pain in the belly, fever, blood or pus in the stool, nausea, dehydration, loss of appetite, loss of weight, etc.). Regardless of initial onset or relapse/recurrence, CDI and/or CDAD may also be diagnosed via enzyme immunoassays, e.g., to detect the C. difficile toxin A or B antigen and/or glutamine dehydrogenase (GDH), which is produced by C. difficile organisms), polymerase chain reactions (e.g., to detect the C. difficile toxin A or B gene or a portion thereof (e.g. tcdA or tcdB), including the ILLUMIGENE LAMP assay), a cell cytotoxicity assay. For example, any of the following tests may be used: Meridian ImmunoCard Toxins A/B; Wampole Toxin A/B Quik Chek; Wampole C. diff Quik Chek Complete; Remel Xpect Clostridium difficile Toxin A/B; Meridian Premier Toxins A/B; Wampole C. difficile Tox A/B II; Remel Prospect Toxin A/B EIA; Biomerieux Vidas C. difficile Toxin A&B; BD Geneohm C. diff; Prodesse Progastro CD; and Cepheld Xpert C. diff. In various embodiments, the clinical sample is a patient stool sample. Also a flexible sigmoidoscopy “scope” test and/or an abdominal X-ray and/or a computerized tomography (CT) scan, which provides images of your colon, may be used in assessing a patient (e.g. looking for characteristic creamy white or yellow plaques adherent to the wall of the colon). Further, biopsies (e.g. of any region of the GI tract) may be used to assess a potential CDI and/or CDAD patient.

In some embodiments, the methods and uses of the present invention include those in which an initial and/or adjunctive therapy is administered to a subject. Initial and/or adjunctive therapy indicates therapy that is used to treat, for example, a microbiome-mediated disorder or disease upon detection of such disorder or disease. In an embodiment, initial and/or adjunctive therapy indicates therapy that is used to treat CDI and/or CDAD upon detection of such disease. In some embodiments, the initial and/or adjunctive therapy is one or more of metronidazole, vancomycin, fidaxomicin, rifaximin, charcoal-based binder/adsorbent, fecal bacteriotherapy, probiotic therapy, and antibody therapy. In various embodiments, the methods and uses of the present invention include use of the alkaline phosphatase as an adjuvant to any of these initial and/or adjunctive therapies (including co-administration or sequential administration). In various embodiments, the methods and uses of the present invention include administration of the AP-based agent described herein to a subject undergoing initial and/or adjunctive therapies.

In various embodiments, the alkaline phosphatase of the invention is administered to a subject who suffers from an increased mucosal permeability of the GI tract. In some embodiments, increased mucosal permeability of the GI tract is the result of a decreased perfusion or ischemia of the intestines. Ischemia, or a lack of oxygen supply by the bloodstream, may be caused by, for example, heart failure, congenital heart disease, congestive heart failure, coronary heart disease, ischemic heart disease, injuries, trauma or surgery. In an embodiment, the AP-based agent is administered to a subject who suffers from leaky gut syndrome.

In some embodiments, the increased mucosal permeability of the GI tract is associated with or caused by autoimmune and inflammatory bowel diseases (IBD), for example, Celiac's disease, Crohn's disease, and colitis (e.g., ulcerative colitis). Accordingly, in some embodiments, the present invention provides methods for treating or preventing autoimmune and IBD, for example, Celiac disease, Crohn's disease, and colitis (e.g., ulcerative colitis), comprising administering an effective amount of an AP-based agent of the invention to a subject in need thereof. IBD is a group of inflammatory conditions of the large intestine and, in some cases, the small intestine. The main forms of IBD are Crohn's disease and ulcerative colitis (UC). IBD also includes collagenous colitis, lymphocytic colitis, ischemic colitis, diversion colitis, Behçet's syndrome, infective colitis, and indeterminate colitis.

Celiac Disease

In some embodiments, the present invention provides methods of treating Celiac disease. In some embodiments, the present invention provides methods of treating GI disorders associated with Celiac disease. Celiac disease is an autoimmune disorder that can occur in genetically predisposed people where the ingestion of gluten leads to damage in the small intestine. Individuals with celiac disease have increased intestinal permeability, which allows gluten break-down products (the triggering antigens of Celiac disease) to reach gut-associated lymphoid tissue, thus initiating an inflammatory response including inflammatory cytokine release and T-cell recruitment. Celiac disease is characterized by chronic inflammation of the small intestinal mucosa that may result in atrophy of the small intestinal villi and diverse symptoms, such as malabsorption, diarrhea, abdominal pain, bloating, fatigue, and nausea. In various embodiments, methods of the invention effectively treat one or more symptoms of Celiac disease including GI symptoms, abdominal symptoms, and non-GI symptoms.

Methods for measuring the improvement in one or more symptoms of Celiac disease can include assessment of the lactulose-to-mannitol (LAMA) ratio, which is an experimental biomarker of intestinal permeability (Kelly et al., (2012) Aliment Pharmacol Ther 2013; 37: 252-262, the entire disclosure is hereby incorporated by reference); measurement of anti-transglutaminase antibody levels; and assessment of clinical symptoms using the Celiac Disease Patient Reported Outcome (CeD PRO), Gastrointestinal Symptom Rating Scale (GSRS), Celiac Disease Gastrointestinal Symptom Rating Scale (CeD GSRS), Bristol Stool Form Scale (BSFS), General Well-Being Questionnaire, Short Form 12 Health Survey Version 2 (SF12V2), Celiac Disease Quality of Life Questionnaire (CeD-QoL), and Clinician Global Assessment of Disease Activity (CGA) as disclosed, for example, in WO/2015/154010, the entire disclosure of which is hereby incorporated by reference. In various embodiments, the present methods of treating Celiac disease provide for a therapeutic effect as assessed by one or more of these measurements.

In some embodiments, the present methods treat Celiac disease and allow a subject to introduce gluten into their diet without substantial symptoms.

AIDS Treatment

In some embodiments, the increased mucosal permeability of the GI tract is associated with or caused by Acquired Immunodeficiency Syndrome (AIDS). Accordingly, in some embodiments, the present invention provides methods of treating GI disorders associated with AIDS. GI disorders are among the most frequent complaints in patients with human immunodeficiency virus 1 (HIV-1) or human immunodeficiency virus 2 (HIV-2)-associated AIDS. GI manifestations of HIV disease include diarrhea, dysphagia, odynophagia, nausea, vomiting, weight loss, abdominal pain, anorectal disease, jaundice, hepatomegaly, GI tract bleeding, and GI tumors (e.g., Kaposi's sarcoma and non-Hodgkin's lymphoma).

Progressive HIV infection often results in GI tract damage, microbial translocation, inflammation, and immune activation which drive progression of disease to AIDS. The term “HIV enteropathy” has been used to describe changes in mucosal structure and function associated with gut-mediated immune dysfunction, as well as to denote the clinical syndrome of chronic diarrhea without an identified infectious cause. In addition to chronic diarrhea, HIV enteropathy is often characterized by increased G1 inflammation, increased intestinal permeability, and malabsorption of bile acids and vitamin B12—abnormalities that are thought to be due to direct or indirect effects of HIV on the enteric mucosa (Brenchley JM, Douek DC. Mucosal Immunol 2008;1:23-30). Clinical consequences include decreased fat and carbohydrate absorption, a trend toward decreased small-bowel transit time, and jejunal atrophy. In various embodiments, methods of the invention effectively treat the symptomatic effects of HIV enteropathy. In various embodiments, methods of the invention prevent, slow, or reverse the progression of HIV infection to AIDS. In various embodiments, methods of the invention prevent or slow the progression of AIDS to death.

Further still, the HIV-1 subtype that a subject becomes infected with may be a factor in the rate of progression to AIDS. In various embodiments, the present methods effectively treat a patient infected with HIV-1 subtype C, D, and G. In another embodiment, the present methods effectively treat a patient infected with HIV-1 subtype A.

In some embodiments, the present invention provides methods of treating various GI disorders associated with HIV infection and/or AIDS. For example, the present invention provides methods of treating HIV-mediated gut dysbiosis and GI barrier dysfunctions, which in various embodiments, may be caused by the HIV, the antibiotics administered to the HIV infected subject, and/or the medications being administered to the HIV infected subject. For example, the HIV infected subject may be taking one or more nucleoside analogues such as deoxyadenosine analogues (e.g., didanosine, vidarabine), adenosine analogues (e.g., BCX4430), deoxycytidine analogues (e.g., cytarabine, emtricitabine, lamivudine, zalcitabine), guanosine and deoxyguanosine analogues (e.g., abacavir, aciclovir, entecavir), thymidine and deoxythymidine analogues (e.g., stavudine, telbivudine, zidovudine), and deoxyuridine analogues (e.g., idoxuridine, trifluridine). In some embodiments, the HIV infected subject may be taking one or more drugs of the highly active anti-retroviral therapy (HAART) regimen. Illustrative HAART medications include entry inhibitors or fusion inhibitors (e.g., maraviroc, enfuvirtide), nucleoside reverse transcriptase inhibitors (NRTI) and nucleotide reverse transcriptase inhibitors (NtRTI) such as the nucleoside and nucleotide analogues described herein, non-nucleoside reverse transcriptase inhibitors (e.g., nevirapine, efavirenz, etravirine, rilpivirine), integrase inhibitors (e.g., raltegravir), and protease inhibitors (e.g., lopinavir, indinavir, nelfinavir, amprenavir, ritonavir, darunavir, atazanavir).

In various embodiments, the present methods reduce local inflammation, alter composition of the GI microbiota, enhance clearance of products of microbial translocation from the circulation, and repair enterocyte barrier in an HIV infected subject and/or a subject having AIDS. In an embodiment, the present methods reduce GI tract damage and gut dysbiosis in an HIV infected subject and/or a subject having AIDS. For example, the present methods may reverse the changes in GI microbiota observed in HIV infected subjects or subjects having AIDS. By way of example, these changes in GI microbiota that may be reversed by the present methods include an altered microbiota featuring increased pathobionts such as Staphylococcus spp., Psedomonas spp., Enterobacteriaceae family members with pro-inflammatory potential, as well as enteropathogenic bacteria that catabolize tryptophan into kynurenine derivatives (including Psudemonas, Xanthomonas, Bacillus, and Burkholderia spp.) In an embodiment, the present methods reduce GI barrier dysfunctions in an HIV infected subject and/or a subject having AIDS. For example, the present methods may reverse the increased intestinal permeability (e.g., leaky gut syndrome) in an HIV infected subject and/or a subject having AIDS. In an embodiment, the present methods reduce microbial translocations or translocations of microbial products and inflammatory mediators (e.g., LPS) into the systemic circulation in an HIV infected subject and/or a subject having AIDS. In such methods, the levels of LPS, EndoCAb, sCD14, and I-FABP in the subject's plasma may be reduced. In an embodiment, the present methods reduce immune activation and inflammation (e.g., local and systemic immune activation and inflammation) in an HIV infected subject and/or a subject having AIDS. For example, the present methods may decrease inflammation in the gut-associated lymphoid tissue (GALT) and increase the number of CD4+cells and Th17 cells. The present methods may further inhibit the release of cytotoxic T cells as well as the production of inflammatory mucosal cytokines and markers such as interferon-a, tumor necrosis factor-α, CRP, IL-1, IL-2, IL-4, IL-6 and IL-13.

Cystic Fibrosis

In some embodiments, the present invention provides methods for treating or preventing dysbiosis and GI dysfunction in patients with cystic fibrosis (CF). The genetic disease CF is associated with mutations in the CF transmembrane conductance regulator (CFTR), which regulates epithelial cell ion and water permeability. In some embodiments, the present methods are used to treating a subject who is homozygous for one or more mutations in the CFTR gene. In some embodiments, the subject is heterozygous for one or more mutations in the CFTR gene. In some embodiments, the one or more CFTR mutations are nonsense mutations. In some embodiments, the one or more CFTR mutations are gating mutations. In some embodiments, the one or more CFTR mutations are protein processing mutations. In some embodiments, the one or more CFTR mutations are conductance mutations. In some embodiments, the one or more CFTR mutations are translation mutations. Examples of CFTR mutations include, but are not limited to, F508del, G542X, G85E, R334W, Y122X, G551D, R117H, A455E, S549R, R553X, V520F, R1162X, R347H, N1203K, S549N, R347P, R560T, G1244E, G1349D, G178R, G551S, S1251N, S1255P, S549R, S1255X, Add9T, Y1092X, M1191K, W 1282X, 3659delC, 394delTT, 3905insT, 1078delT, delta 1507, 3876delA, 2184delA, 2307insA, 711+1G>T, 1717-1G>A, 2789+5G>A, 1898+5G>T, 3120+1G>A, 621+1G>T, 3849+10kbC>T, 1898+1G>A, 2183 AA>G, and/or 5/7/9T. In various embodiments, methods of the invention are used to treat a CF patient having one or more of the CFTR mutations disclosure herein. In an embodiment, the patient has one or more of the following CFTR mutations: G551D, G1244E, G1349D, G178R, G551S, S1251N, S1255P, S549N, S549R and/or R117H. In an embodiment, the patient has a F508del mutation. Methods for screening a patient's genotype for CFTR mutations are known and may be carried out by, for example, DNA sequencing such as bidirectional sequencing.

CF patients often exhibit symptoms including chronic respiratory infections and dysfunction at GI mucosal surfaces, resulting insubstantial morbidity and mortality. One of the earliest manifestations of CF is GI dysfunction including severe and recurrent intestinal obstruction as well as nutrient malabsorption, which result in growth failure. CF patients also exhibit GI dysbiosis such as an overabundance of E. coli in the fecal microbiota and a decrease in the relative abundance of Bifidobacterium species. In various embodiments, methods of the invention effectively treat one or more GI-related symptoms of in CF patients.

Methods for measuring change and/or improvement in G1 tract function can include, but are not limited to: endoscopy for direct examination of epithelium and mucosa; histological evaluation and/or tissue procurement for direct evaluation of structural changes and/or immune biomarkers; urine tests for assessment of permeability with non-absorbable sugars and LPS levels; stool tests for assessment of inflammation and/or microbiota changes (for example by PCR); and/or blood tests for assessment of specific markers, including CD4+ cell counts, Th17 cell counts, and/or LPS levels.

In some embodiments, the present invention provides methods of treating GI disorders associated with hypothyroidism. Hypothyroidism is a condition in which the thyroid gland does not produce enough thyroid hormone (thyroxine or T4). Often, hypothyroidism slows the actions of the digestive tract causing constipation, or the digestive tract may stop moving entirely. Methods of the invention may alleviate the one or more GI symptoms associated with hypothyroidism.

NEC Treatment

In one aspect, the present invention provides methods for preventing or treating necrotizing enterocolitis (NEC). The present methods comprise administering to a subject in need thereof an AP-based agent as described herein or a pharmaceutical composition or a formulation such as a modified-release formulation as described herein.

In various embodiments, methods of the invention relate to a pediatric subject for the prevention or treatment of NEC. In various embodiments, the pediatric subject may be from about 1 day to about 1 week old, from about 1 week to about 1 month old, from about 1 month to about 12 months old, from about 12 months to about 18 months old, from about 18 to about 36 months old, from about 1 to about 5 years old, from about 5 to about 10 years old, from about 10 to about 15 years old, or from about 15 to about 18 years old. In some embodiments, the pediatric subject is an infant of about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, or about 12 months of age. In various embodiments, the pediatric subject is feeding on formula and/or milk. In various embodiments, the pediatric subject is undergoing treatment or has recently undergone treatment with an antibiotic.

In various embodiments, the pediatric subject is a premature infant. In some embodiments, the premature infant is born at less than 37 weeks of gestational age. In some embodiments, the premature infant is born at about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, or about 37 weeks of gestational age. In other embodiments, the pediatric subject is a full term infant, for example, an infant who is born later than about 37 weeks of gestational age. In some embodiments, the pediatric subject may exhibit one or more of prenatal asphyxia, shock, sepsis, or congenital heart disease. In various embodiments, the pediatric subject is of low birth weight. In various embodiments, the pediatric subject weighs less than about 5 pounds, about 4 pounds, about 3 pounds, or about 2 pounds.

In various embodiments, methods of the invention relate to a pregnant woman for the prevention or treatment of NEC. In some embodiments, the pregnant woman is undergoing treatment or has recently undergone treatment with an antibiotic.

The presence and severity of NEC is graded using the staging system of Bell et al., J. Ped. Surg., 15:569 (1980). In various embodiments, the present methods treat disease at any of these stages.

In various embodiments, methods of the invention effectively treat one or more symptoms of NEC including any of the symptoms described above as well as those symptoms known in the art, including GI symptoms, abdominal symptoms, and non-GI symptoms. In various embodiments, methods of the invention effectively prevent the development of NEC in a subject such as a pediatric subject. In various embodiments, methods of the invention effectively prevent progression of NEC in a subject such as a pediatric subject, for example, from stage I to stage II or from stage II to stage III. In various embodiments, methods of the invention effectively result in regression of NEC in a subject such as a pediatric subject, for example, from stage III to stage II or stage Ito complete cure, or from stage II to stage I or to complete cure.

Intestinal dysbiosis is associated with the development of NEC and can be detected in a subject prior to any clinical evidence of the disease. In various embodiments, methods of the invention effectively restore normal microbiota in the intestinal tract of the treated subject. In some embodiments, methods of the invention maintain a normal microbiota in the intestinal tract. For instance, in some embodiments, the methods of the invention maintain a healthy balance (e.g. a healthy ratio and/or healthy distribution) of intestinal microbiota of a subject. In another embodiment, the methods of the invention treat or prevent the overgrowth of one or more pathogenic microorganisms in the GI tract. In certain embodiments, methods of the invention effectively reduce the levels of Clostridium butyricum and/or Clostridium perfringens in the intestinal tract.

Methods for measuring the improvement in one or more symptoms of NEC include diagnostic imaging modalities such as X-ray and ultrasonography. Methods for measuring change and/or improvement in GI tract function can include, but are not limited to: endoscopy or colonoscopy for direct examination of epithelium and mucosa; histological evaluation and/or tissue procurement for direct evaluation of structural changes and/or immune biomarkers; stool tests for assessment of inflammation and/or microbiota changes (for example by PCR); and/or blood tests for assessment of specific markers and cells.

In some embodiments, the present invention provides methods of treating or preventing metabolic syndrome, diabetes, hypertension, cardiovascular disease, nonalcoholic fatty liver and other metabolic diseases. In various embodiments, the metabolic syndrome is associated with elevated triglycerides, elevated low density lipoproteins, reduced high density lipoproteins, reduced lipoprotein index, elevated fasting glucose levels, elevated fasting insulin, reduced glucose clearance following feeding, insulin resistance, impaired glucose tolerance, obesity and combinations thereof. For example, the present methods may be used to treat subjects having metabolic syndrome and having abdominal obesity (e.g., waist circumference of 40 inches or above in men or 35 inches or above in women), a blood triglyceride level of 150 mg/dL or greater, HDL of less than 40 mg/dL in men or less than 50 mg/dL in women, systolic blood pressure of 130 mm Hg or greater or diastolic blood pressure of 85 mm Hg or greater and/or fasting glucose of 100 mg/dL or greater. Additional metabolic diseases that may be treated using methods of the invention include those described in US2013/0251701, US2011/0206654, and US2004/0115185, the entire contents of which are hereby incorporated by reference.

In an embodiment, the metabolic disease is obesity. Early exposure to antibiotics (e.g. within about the first 2 years of life) can disrupt the microbiome and lead to eventual disease. Bailey, et al. JAMA Pediatr. 168(11), November 2014, the entire contents of which are hereby incorporated by reference, describes how early exposure to antibiotics is linked to obesity. Accordingly, in some embodiments, the present methods protect the microbiome of a child and prevent diseases such as obesity. In addition, a shift in the ratio between bacterial divisions Firmicutes and Bacteroidetes is often observed in obese individuals. Accordingly, in some embodiments, the present invention provides methods for treating or preventing obesity by administering an AP agent of the invention. Methods of the invention retain a normal diversity of bacteria in the intestinal tract, such as for example, Bacteroidetes, Proteobacteria, and Firmicutes, thereby treating or preventing obesity. Further still, alkaline phosphatases may influence fat absorption at the GI tract. Accordingly, in various embodiments, the present invention provides methods for treating or preventing obesity by limiting GI fat absorption. In various embodiments, methods of the invention are effective for inducing weight loss or preventing weight gain. In some embodiments, the subjects may have undertaken or will undertake a surgery of the digestive system; be greater than about 80-100 pounds overweight; have a BMI of greater than about 35 kg/m2; or have a health problem related to obesity. In some embodiments, the subjects may have dyslipidemia including hyperlipidemia and hyperlipoproteinemia.

Diabetes

In another embodiment, the metabolic disease is diabetes. In various embodiments, the present invention relates to the treatment for diabetes (type 1 or type 2) and/or glucose intolerance. In some embodiments, the present invention relates to a method for treating subjects at risk of diabetes, one or more of insulin resistance, prediabetes, impaired fasting glucose (IFG), and impaired glucose tolerance (IGT).

In various embodiments, the present invention relates to the treatment of type 1 diabetes with AP, including the formulations described herein. Type 1 diabetes, once known as juvenile diabetes or insulin-dependent diabetes, is a chronic condition in which the pancreas produces little or no insulin. Treatment is often via intensive insulin regimens, which attempt to mimic the body's normal pattern of insulin secretion, and often involve basal and bolus insulin coverage. For example, one common regimen is the administration of a long-acting insulin (including, for example, glargine/detemir) once or twice a day with rapid acting insulin (including, for example, aspart, glulisine, lispro) preprandially or postprandially and as needed to correct high blood sugars (as monitored by a glucose meter, for example). Doses administered preprandially or postprandially or as needed to correct high blood sugars may be referred to as bolus administrations. Another common regimen involves dosing, including continuous dosing, via an insulin pump (or continuous subcutaneous insulin infusion device (CSII) of, for example a rapid acting insulin (as described herein and including, for example, aspart, glulisine, lispro). In various embodiments, AP, including the formulations described herein, may replace any of the insulins used in various regimens, including instances in which the insulins are not providing effective therapy in the patient. AP, including the formulations described herein, may cause an increase in patient compliance as it may allow for easier self-dosing relative to various forms of insulin, which must be administered as various doses throughout the day—even in the context of an insulin pump, which requires programming. Further, AP, including the formulations described herein, can offset common frustration of diabetic patient dosing, such as, for example, the dawn phenomenon. Alternatively, AP, including the formulations described herein, may be used adjuvant to any of the type 1 diabetes treatments described herein to, for example, normalize a patient's regimen and avoid blood sugar “dips” (e.g. hypoglycemia, e.g. blood sugar of below about 70 mg/dL) and “spikes” (e.g. hyperglycemia, e.g. blood sugar of greater than about 200 mg/dL) that afflict many patients. Accordingly, in some embodiments, AP, including the formulations described herein, may treat or prevent symptoms associated with hypoglycemia, including for example, shakiness, anxiety, nervousness, palpitations, tachycardia, pallor, coldness, clamminess, dilated pupils (mydriasis), hunger, borborygmus, nausea, vomiting, abdominal discomfort, headache, abnormal mentation, impaired judgment, nonspecific dysphoria, paresthesia, negativism, irritability, belligerence, combativeness, rage, personality change, emotional lability, fatigue, weakness, apathy, lethargy, daydreaming, sleep, confusion, amnesia, lightheadedness or dizziness, delirium, staring, “glassy” look, blurred vision, double vision, flashes of light in the field of vision, automatism, difficulty speaking, slurred speech, ataxia, incoordination, focal or general motor deficit, paralysis, hemiparesis, paresthesia, headache, stupor, coma, abnormal breathing, generalized or focal seizures, memory loss, CNS damage (e.g. cognitive impairment), amnesia, and death. Accordingly, in some embodiments, AP, including the formulations described herein, may treat or prevent symptoms associated with hyperglycemia, including for example, polyphagia, polydipsia, polyuria, blurred vision, fatigue, weight loss, poor wound healing, dry mouth, dry or itchy skin, tingling in feet or heels, erectile dysfunction, recurrent infections, external ear infections (e.g. swimmer's ear), cardiac arrhythmia, stupor, coma, and seizures. In various regimens, a type 1 diabetes patient may receive additional agents to supplement insulin therapy. In some embodiments, AP, including the formulations described herein, are used in this manner. AP, including the formulations described herein, may provide additional therapeutic benefits in patients that are struggling to manage type 1 diabetes with insulin therapy alone. In some embodiments, patients that are struggling to manage type 1 diabetes with insulin therapy alone have poor glycemic control as described herein.

In some embodiments, AP, including the formulations described herein, finds use in reducing a patient's blood glucose level to below about 10 mM, e.g. within the range of about 4 mM to about 7 mM.

In some aspects, the present invention provides a method for treating type 1 or type 2 diabetes, comprising administering an effective amount of AP, including the formulations described herein.

In a number of embodiments, including those in which AP, including the formulations described herein, prevents diabetes and/or treats a pre-diabetic condition, a patient is at risk of diabetes if the patient is characterized by one or more of: being physically inactive; having a parent or sibling with diabetes; having a family background associated with high incidence of diabetes, selected from that is African American, Alaska Native, American Indian, Asian American, Hispanic/Latino, or Pacific Islander American; giving birth to a baby weighing more than 9 pounds; being diagnosed with gestational diabetes; having high blood pressure of about 140/90 mmHg or above; being treated for high blood pressure; having HDL cholesterol level below about 35 mg/dL and/or a triglyceride level above about 250 mg/dL; having polycystic ovary syndrome (PCOS); and having cardiovascular disease.

In various embodiments, AP, including the formulations described herein, may be used to treat diabetes in the context of hospitalization. For example, in some embodiments, AP, including the formulations described herein, may be administered to a patient that is in a diabetic coma. In some embodiments, the patient may be administered to a patient that has one or more of a severe diabetic hypoglycemia, advanced diabetic ketoacidosis (e.g. advanced enough to result in unconsciousness, contributing factors may include one or more of hyperglycemia, dehydration, shock, and exhaustion), hyperosmolar nonketotic coma (e.g. with one or more of hyperglycemia and dehydration are contributing factors). In these embodiments, AP, including the formulations described herein, may be used in conjunction with standard treatment regimens of diabetic comas, including administering one or more of glucose, glucagon, insulin, fluids (e.g. saline with potassium and/or other electrolytes), any of which, optionally, are administered intravenously. In some embodiments, AP, including the formulations described herein, may replace insulin in these treatment regimens and, optionally, is administered orally.

Further, in various embodiments pertaining to diabetes, the patient may be recieving or there may be co-administration with one or more additional agents. Illustrative additional agents include insulin or any anti-diabetic agents (e.g. biguanides, insulin secretogogues such as sulphonylureas or meglitinides, inhibitors of α-glucosidase, thiazolidinediones, and others). The methods of treatment described herein, in various embodiments may comprise administering AP, including the formulations described herein, to a patient that is receiving one or more additional agents and/or non-insulin diabetes agents. Additional agents include one or more of a sulfonylurea (e.g. DYMELOR (acetohexamide), DIABINESE (chlorpropamide), ORINASE (tolbutamide), and TOLINASE (tolazamide), GLUCOTROL (glipizide), GLUCOTROL XL (extended release), DIABETA (glyburide), MICRONASE (glyburide), GLYNASE PRESTAB (glyburide), and AMARYL (glimepiride)); a Biguanide (e.g. metformin (GLUCOPHAGE, GLUCOPHAGE XR, RIOMET, FORTAMET, and GLUMETZA); a thiazolidinedione (e.g. ACTOS (pioglitazone) and AVANDIA (rosiglitazone); an alpha-glucosidase inhibitor (e.g., PRECOSE (acarbose) and GLYSET (miglitol); a Meglitinide (e.g., PRANDIN (repaglinide) and STARLIX (nateglinide); a Dipeptidyl peptidase IV (DPP-IV) inhibitor (e.g., JANUVIA (sitagliptin), NESINA (alogliptin), ONGLYZA (saxagliptin), and TRADJENTA (linagliptin); Sodium-glucose co-transporter 2 (SGLT2) inhibitor (e.g. INVOKANA (canaglifozin)); and a combination pill (e.g. GLUCOVANCE, which combines glyburide (a sulfonylurea) and metformin, METAGLIP, which combines glipizide (a sulfonylurea) and metformin, and AVANDAMET, which uses both metformin and rosiglitazone (AVANDIA) in one pill, KAZANO (alogliptin and metformin), and OSENI (alogliptin plus pioglitazone).

Other additional agents include METFORMIN oral, ACTOS oral, BYETTA subcutaneous, JANUVIA oral, WELCHOL oral, JANUMET oral, glipizide oral, glimepiride oral, GLUCOPHAGE oral, LANTUS subcutaneous, glyburide oral, ONGLYZA oral, AMARY1 oral, LANTUS SOLOSTAR subcutaneous, BYDUREON subcutaneous, LEVEMIR FLEXPEN subcutaneous, ACTOPLUS MET oral, GLUMETZA oral, TRADJENTA oral, bromocriptine oral, KOMBIGLYZE XR oral, INVOKANA oral, PRANDIN oral, LEVEMIR subcutaneous, PARLODEL oral, pioglitazone oral, NOVOLOG subcutaneous, NOVOLOG FLEXPEN subcutaneous, VICTOZA 2-PAK subcutaneous, HUMALOG subcutaneous, STARLIX oral, FORTAMET oral, GLUCOVANCE oral, GLUCOPHAGE XR oral, NOVOLOG Mix 70-30 FLEXPEN subcutaneous, GLYBURIDE-METFORMIN oral, acarbose oral, SYMLINPEN 60 subcutaneous, GLUCOTRO1 XL oral, NOVOLIN R GLUCOTROL oral, DUETACT oral, sitagliptin oral, SYMLINPEN 120 subcutaneous, HUMALOG KWIKPEN subcutaneous, JANUMET XR oral, GLIPIZIDE-METFORMIN oral, CYCLOSET oral, HUMALOG MIX 75-25 subcutaneous, nateglinide oral, HUMALOG Mix 75-25 KWIKPEN subcutaneous, HUMULIN 70/30 subcutaneous, PRECOSE oral, APIDRA subcutaneous, Humulin R inj, Jentadueto oral, Victoza 3-Pak subcutaneous, Novolin 70/30 subcutaneous, NOVOLIN N subcutaneous, insulin detemir subcutaneous, glyburide micronized oral, GLYNASE oral, HUMULIN N subcutaneous, insulin glargine subcutaneous, RIOMET oral, pioglitazone-metformin oral, APIDRA SOLOSTAR subcutaneous, insulin lispro subcutaneous, GLYSET oral, HUMULIN 70/30 Pen subcutaneous, colesevelam oral, sitagliptin-metformin oral, DIABETA oral, insulin regular human inj, HUMULIN N Pen subcutaneous, exenatide subcutaneous, HUMALOG Mix 50-50 KWIKPEN subcutaneous, liraglutide subcutaneous, KAZANO oral, repaglinide oral, chlorpropamide oral, insulin aspart subcutaneous, NOVOLOG Mix 70-30 subcutaneous, HUMALOG Mix 50-50 subcutaneous, saxagliptin oral, ACTOPLUS Met XR oral, miglitol oral, NPH insulin human recomb subcutaneous, insulin NPH and regular human subcutaneous, tolazamide oral, mifepristone oral, insulin aspart protam-insulin aspart subcutaneous, repaglinide-metformin oral, saxagliptin-metformin oral, linagliptin-metformin oral, NESINA oral, OSENI oral, tolbutamide oral, insulin lispro protamine and lispro subcutaneous, pramlintide subcutaneous, insulin glulisine subcutaneous, pioglitazone-glimepiride oral, PRANDIMET oral, NOVOLOG PenFill subcutaneous, linagliptin oral, exenatide microspheres subcutaneous, KORLYM oral, alogliptin oral, alogliptin-pioglitazone oral, alogliptin-metformin oral, and canagliflozin oral.

Other additional agents include Lispro (HUMALOG); Aspart (NOVOLOG); Glulisine (APIDRA); Regular (NOVOLIN R or HUMULIN R); NPH (NOVOLIN N or HUMULIN N); Glargine (LANTUS); Detemir (LEVEMIR); HUMULIN or NOVOLIN 70/30; and NOVOLOG Mix 70/30 HUMALOG Mix 75/25 or 50/50.

Neurological Disease

In various embodiments, the present invention is used to treat or prevent various neurodegenerative diseases. In some embodiments, the neurodegenerative disease is selected from multiple sclerosis (MS; including, without limitation benign multiple sclerosis, relapsing-remitting multiple sclerosis (RRMS), secondary progressive multiple sclerosis (SPMS), progressive relapsing multiple sclerosis (PRMS), and primary progressive multiple sclerosis (PPMS), Alzheimer's. disease (including, without limitation, Early-onset Alzheimer's, Late-onset Alzheimer's, and Familial Alzheimer's disease (FAD), Parkinson's disease and parkinsonism (including, without limitation, Idiopathic Parkinson's disease, Vascular parkinsonism, Drug-induced parkinsonism, Dementia with Lewy bodies, Inherited Parkinson's, Juvenile Parkinson's), Huntington's disease, Amyotrophic lateral sclerosis (ALS, including, without limitation, Sporadic ALS, Familial ALS, Wesrtem Pacific ALS, Juvenile ALS, Hiramaya Disease).

Sepsis

In various embodiments, the present invention provides methods of treating or preventing sepsis. Sepsis is characterized by a whole-body inflammatory state caused by infection. Sepsis includes the presence of various pus-forming and other pathogenic organisms, or their toxins, in the blood or tissues. In some embodiments, the present invention provides methods of treating or preventing septicemia (blood poisoning), bacteremia, viremia, and/or fungemia. In various embodiments, the present invention treats the various end-organ pathologies associated with sepsis such as hypotension, acute tubular necrosis (ATN) and acute respiratory distress syndrome (ARDS).

Acute Kidney Injury and Renal Failure

In various embodiments, the present invention provides methods of treating or preventing acute kidney injury (AM). Acute kidney injury (formerly known as acute renal failure) is a severe inflammation and damage of the kidney, which sometimes results in complete kidney failure. AM is characterized by the rapid loss of the kidney's excretory function and is typically diagnosed by the accumulation of end products of nitrogen metabolism (urea and creatinine) or decreased urine output, or both. It is the clinical manifestation of several disorders that affect the kidney acutely. Patients who have had acute kidney injury are at increased risk of developing chronic kidney disease. In some embodiments, the acute kidney injury is an ischemic acute kidney injury.

In various embodiments, the present invention provides methods of treating or preventing renal failure such as acute renal failure (ARF). Acute renal failure involves an acute loss of kidney function that results in an increase of the serum creatinine level. In acute renal failure, the glomerular filtration rate decreases over days to weeks. As a result, excretion of nitrogenous waste is reduced, and fluid and electrolyte balances cannot be maintained. Patients with acute renal failure are often asymptomatic, and the condition is diagnosed by observed elevations of blood urea nitrogen (BUN) and serum creatinine levels. Complete renal shutdown is present when the serum creatinine level rises by at least 0.5 mg per dL per day and the urine output is less than 400 mL per day (oliguria). The AP-based agents described herein can be used not only in the treatment of renal failure but also to improve renal cases where the renal function is at least partly impaired or reduced.

Radiation-Induced Enteropathy, Colitis, and/or Proctitis

In various embodiments, the present invention provides methods of treating or preventing radiation-induced enteropathy, colitis, and/or proctitis. Radiation-induced enteropathy is characterized by mucosal atrophy, vascular sclerosis, and progressive intestinal wall fibrosis. Symptoms of the disorder can include malabsorption of nutrients, altered intestinal transit, dysmotility, and abnormal propulsion of intestinal contents. In some embodiments, acute radiation-induced enteropathy occurs within the first month, first 2 months, or first 3 months after radiation exposure. In some embodiments, delayed radiation enteropathy symptoms are chronic and may not present until at least 3, at least 4, at least 5, at least 6, at least 7, at least 8, at least 9, at least 10, at least 11, or at least 12 months after radiation exposure. In some embodiments, delayed radiation enteropathy symptoms may not present until about 3, about 4, about 5, about 6, about 7, about 8, about 9, about 10, about 11, or about 12 months after radiation exposure. In some embodiments, delayed radiation enteropathy symptoms may not present until about 1 year, about 2 years, about 3 years, about 4 years, or about 5 years after radiation exposure.

In various embodiments, administration of the AP-based agent occurs prior to exposure to radiation, such as, for example, prior to radiotherapy as part of a cancer treatment. In certain embodiments, administration of the AP-based agent occurs at the time of radiation exposure. In various embodiments, administration of the AP-based agent occurs at the time of exposure to radiation, as well as shortly after exposure to radiation. In some embodiments, administration of the AP-based agent occurs shortly after exposure to radiation. In various embodiments, administration of the AP-based agent occurs at the time of exposure to radiation, as well as continued long term after exposure to radiation. In some embodiments, administration of the AP-based agent continues for a long term after exposure to radiation. In various embodiments, administration of the AP-based agent occurs at the onset of delayed radiation enteropathy. In some embodiments, the present invention provides for the treatment and/or administration of an AP-based agent to a subject that has been exposed to or will be exposed to radiation, where the administration of the AP-based agent occurs for at least 1 year, at least 1.5 years, at least 2 years, at least 2.5 years, at least 3 years, at least 3.5 years, 4 years, at least 4.5 years, at least 5 years, at least 5.5 years, at least 6 years, at least 6.5 years, or at least 7 years after the exposure to radiation.

### EXAMPLES

### Example 1. Production of IAP in a Bioreactor

An IAP-encoding transfected CHO cell line was provided. The cell line was seeded in a 3 L bioreactor (Moebius® CellReady 3 L, Merck Millipore) at a seeding volume of 1.1 L, and the cultures were maintained for a period of 14 days. A total of three different conditions were established and run (see Table 1). Conditions 1 and 2 were run in duplicates, while Condition 3 was not run in duplicate. Seeding density varied among the three conditions, as shown in Table 1 below.

The initial culture medium consisted of EX-CELL® Advanced CHO fed batch (Sigma-Aldrich) supplemented with 4mM Glutamine (Gln), 1×HT Supplement liquid (a mixture of sodium hypoxanthine and thymidine), 80 μM ZnSO₄, 1mM MgCl₂, 0.11% poloxamer (e.g., Kolliphor P188), and 0.1% antifoam. The cultures were fed batch daily with two different feeds (Feed A and Feed B) at varying percentages among the three conditions, starting on day 2 and ending on day 13.

Specifically, Condition 1 was run with a seeding density of 0.5×10⁶ cells/mL, fed batch with 2.8% feed A and 0.28% feed B based on the current volume, and a pH of 6.85. Condition 2 was run with a seeding density of 0.75×10⁶ cells/mL, fed batch with 3.0% feed A and 0.3% feed B based on the current volume, and a pH of 6.85. Finally, Condition 3 was run with a seeding density of 0.5×10⁶ cells/mL, fed batch with 2.8% feed A and 0.28% feed B based on the current volume, a pH of 6.85, and an addition of 80 μM supplemental ZnSO₄ to the culture medium on day 11 of the process.

TABLE 1
Bioreactor culture conditions.

| | | | Feed percentage | | | |
|---|---|---|---|---|---|---|
| | | Seeding density | from day 3 to day | | | Supplementary |
| STR# | Condition | [cells/ml] | 13 | | pH setpoint | Addition of Zn |
| BR2 | #1 | 0.50E06 | 2.8% | feed A | 6.85 ± 0.05 | — |
| | | | 0.28% | feed B | | |
| BR3 | #1 | 0.50E06 | 2.8% | feed A | 6.85 ± 0.05 | — |
| | | | 0.28% | feed B | | |
| BR5 | #2 | 0.75E06 | 3.0% | feed A | 6.85 ± 0.05 | — |
| | | | 0.3% | feed B | | |
| BR6 | #2 | 0.75E06 | 3.0% | feed A | 6.85 ± 0.05 | — |
| | | | 0.3% | feed B | | |
| BR7 | #3 | 0.50E06 | 2.8% | feed A | 6.85 ± 0.05 | On day 11 |
| | | | 0.28% | feed B | | |

Shifts in temperature occurred during the processes of all three conditions. Specifically, a first temperature shift from 37° C. to 33° C. occurred at about 72 hours after the initiation of the culture within the bioreactor. Then, a second temperature shift from 33° C. to 31° C. occurred at about 288 hours after the initiation of the culture within the bioreactor.

At the initiation of the bioreactor culturing process, the pH of the culture medium was allowed to drift until day 3, after which the pH was forced to the setpoint of 6.85±0.05.

For Conditions 1 and 3, Feed A (making up 2.8% feeding) consisted of 64 g/L glucose and 10 mg/L insulin, a carbon source, concentrated amino acids, vitamins, salts, trace minerals and did not contain lipids, hydrolysates, or growth factors.

For Condition 2, Feed A (making up 3.0% feeding) consisted of 60 g/L glucose and 10 mg/L insulin. Feed A also contained a carbon source, concentrated amino acids, vitamins, salts, trace minerals; and did not contain lipids, hydrolysates, or growth factors.

Feed B for all conditions consisted of a carbon source, concentrated amino acids, vitamins, salts, trace minerals. Feed B for all conditions did not contain lipids, hydrolysates, or growth factors.

The CHO cells producing recombinant IAP were separated from the medium on day 13 of the culture process and the IAP protein was recovered at day 14.

### Example 2. Analysis of IAP Produced in Bioreactor

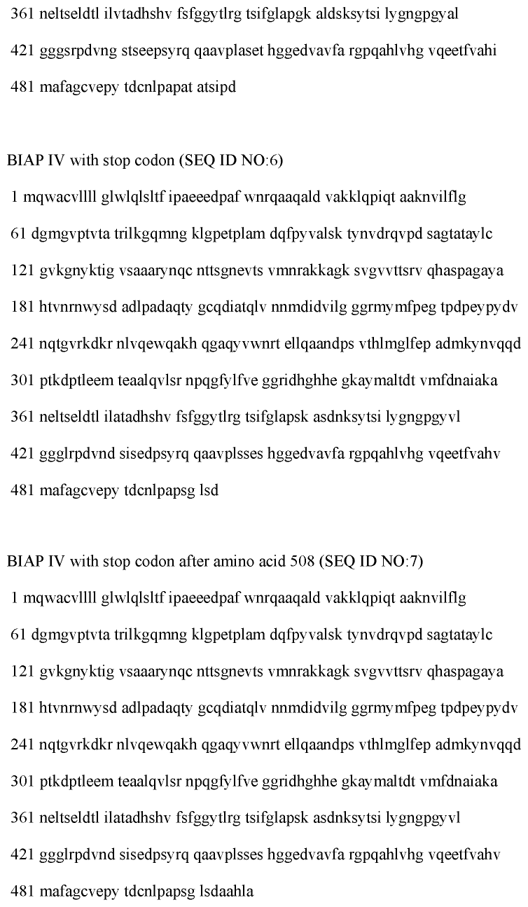

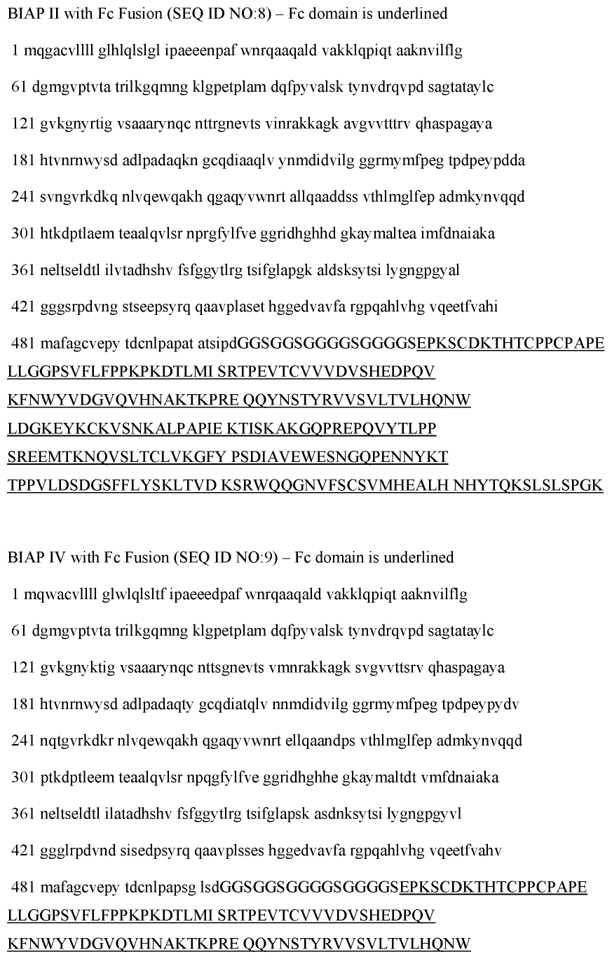

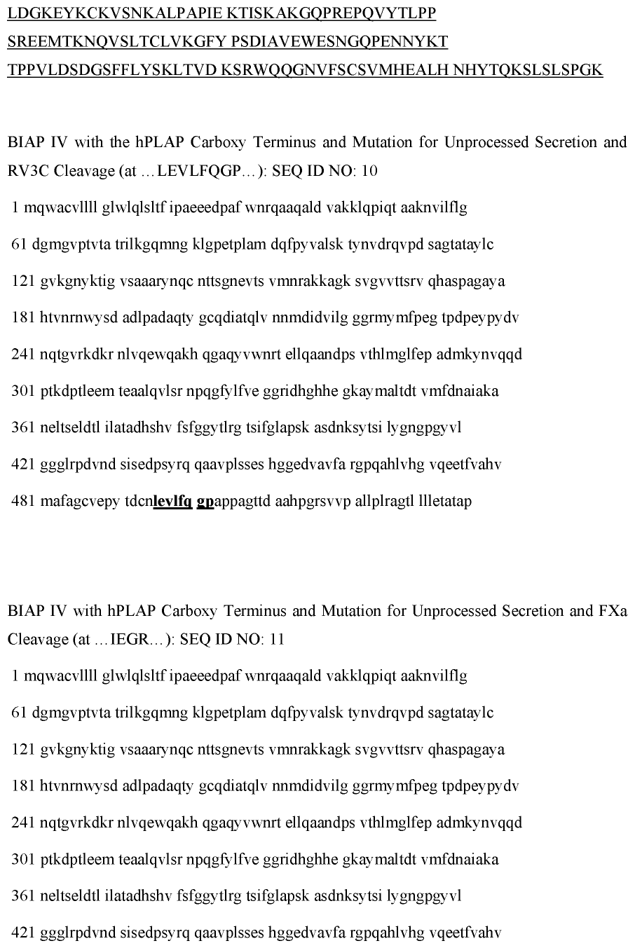

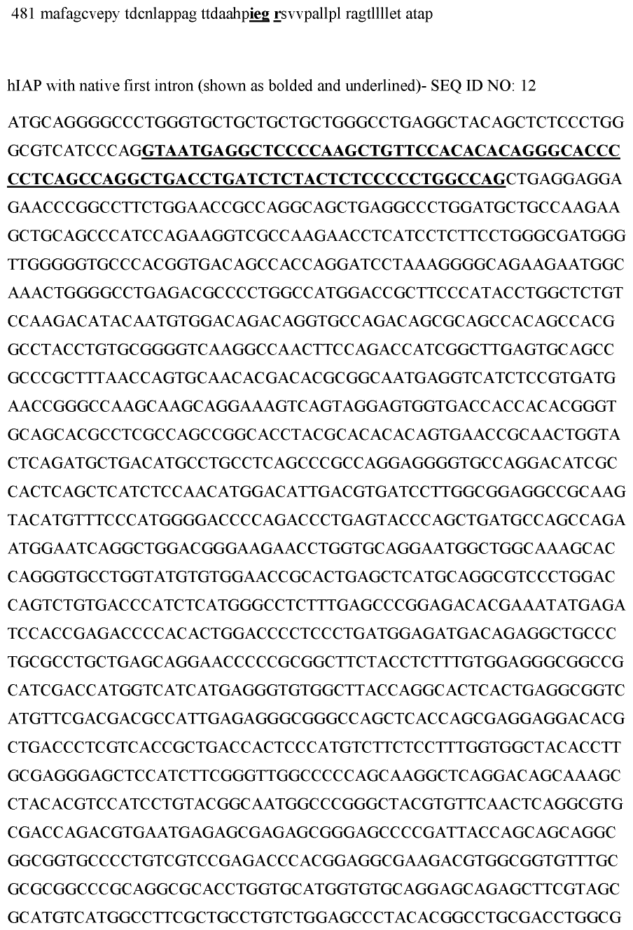

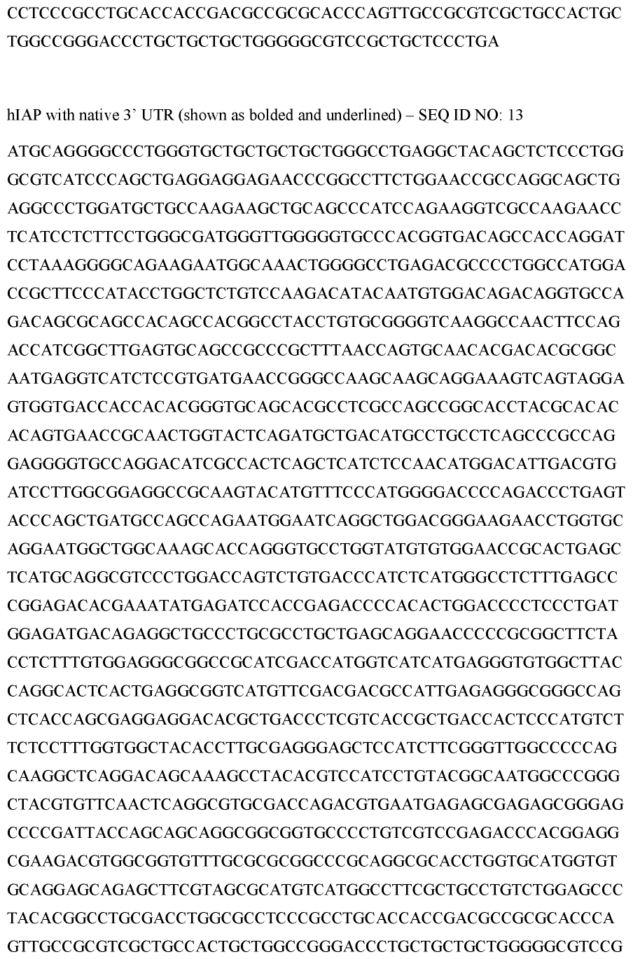

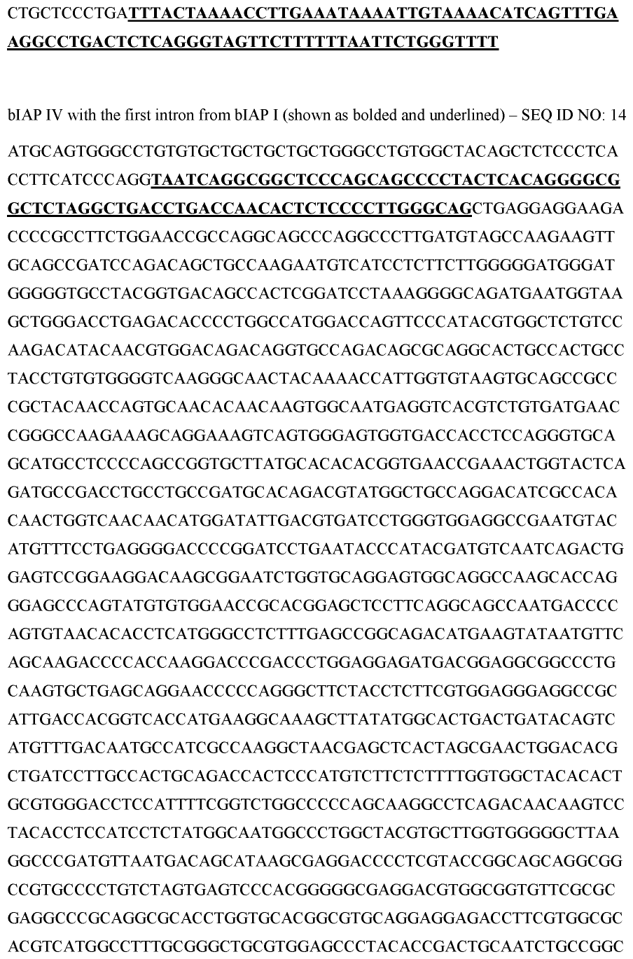

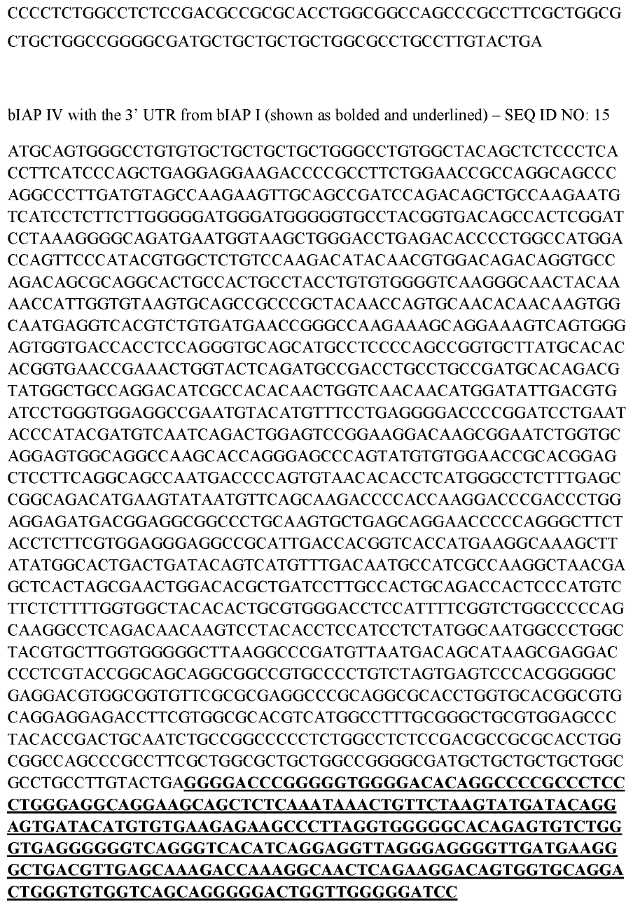

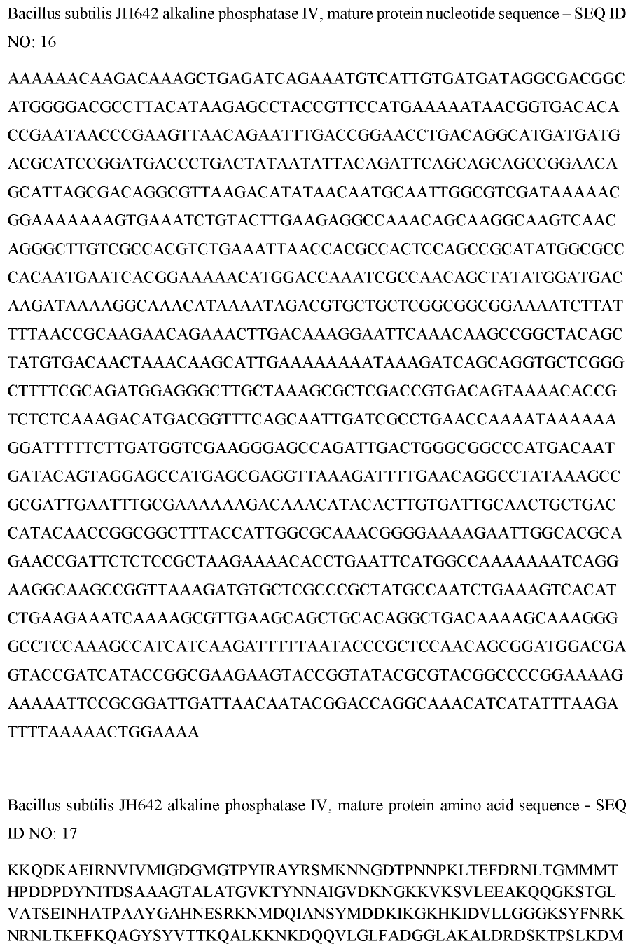

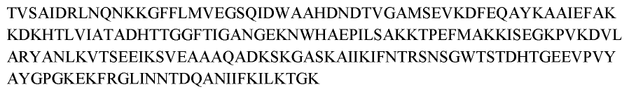

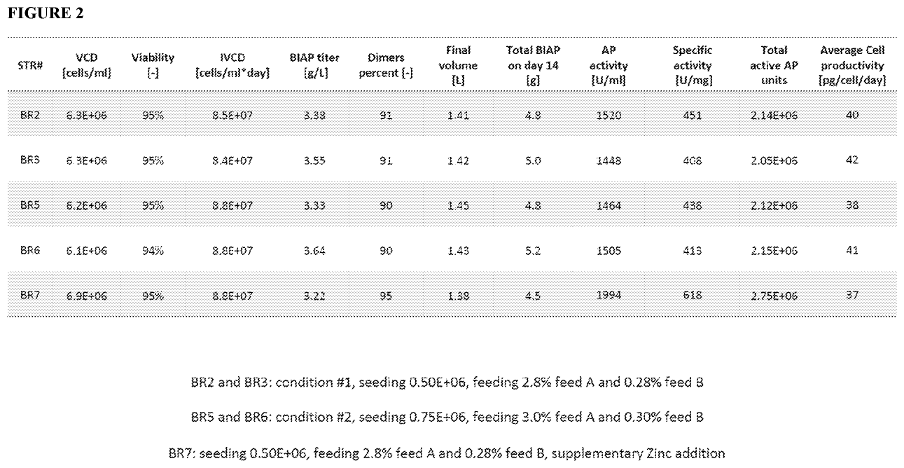

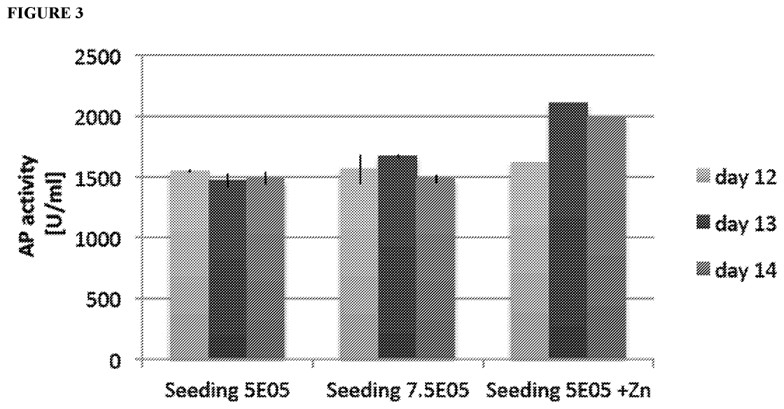

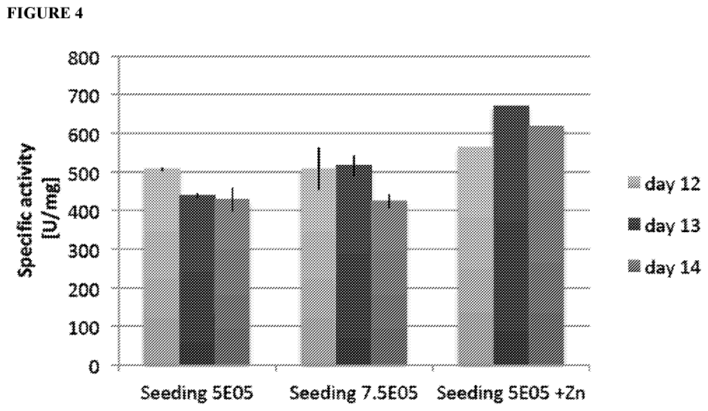

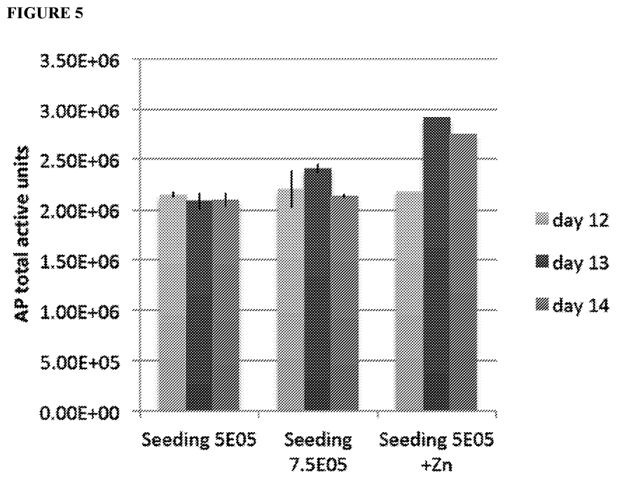

Surprisingly, the IAP produced in the bioreactor process under Condition 3 exhibited higher alkaline phosphatase (AP) activity (U/mL), specific activity (U/mg), and total active AP units, as compared to IAP produced in the bioreactor processes under Conditions 1 and 2. As shown in FIG. 2, the IAP produced from Bioreactor 7 (BR7, under Condition 3) exhibited higher AP activity, specific activity, and total active AP units on Day 14. Similarly, FIG. 3, FIG. 4, and FIG. 5 depict the differences in readings of AP activity, specific activity, and AP total active units, respectively, on days 12, 13, and 14, among IAP produced under all three conditions. For example, FIG. 2 shows that there was about a 37% increase in specific activity and about a 31% increase in AP activity of IAP produced in BR7 (Condition 3, with an addition of 80 μM supplemental ZnSO₄) as compared to BR2 (Condition 1, with no addition of supplemental ZnSO₄).

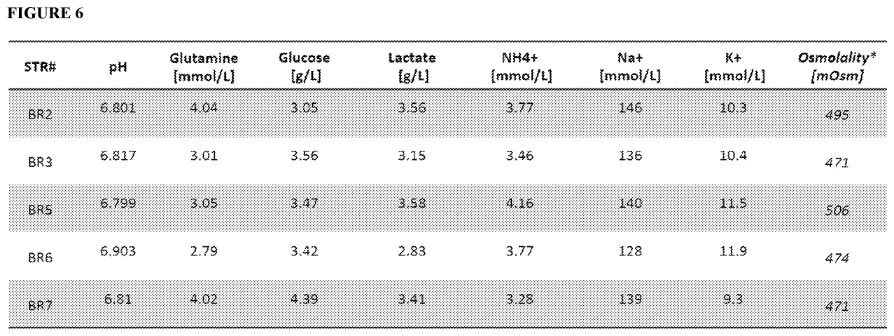

Various metabolites, pH, and osmolality were also measured daily throughout the bioreactor process after the addition of Feed A and Feed B. Metabolite content, such as amount of glutamine, insulin, lactate, ammonium (NH4+), sodium (Na+), and/or potassium (K+), was measured using a BioProfile® 400 (NOVA Biomedical) cell culture analyzer. Osmolality was measured with an osmometer. FIG. 6 depicts metabolite content, pH, and osmolality of day 14 for IAP produced under all three conditions.

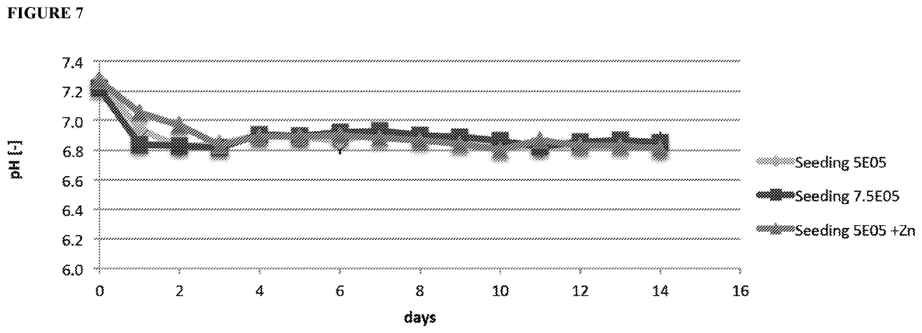

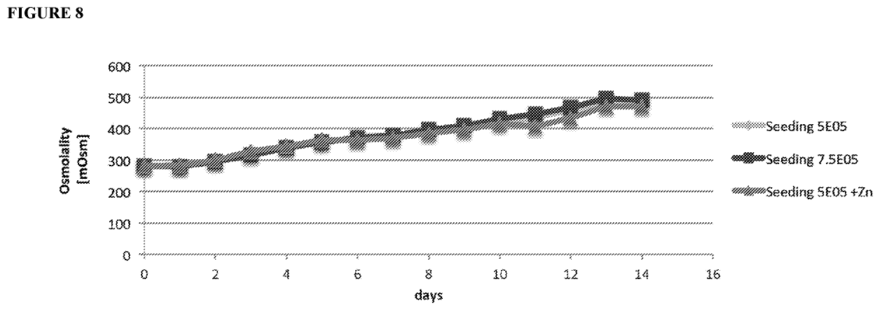

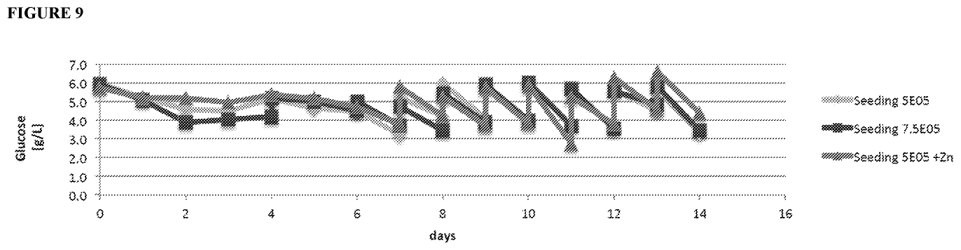

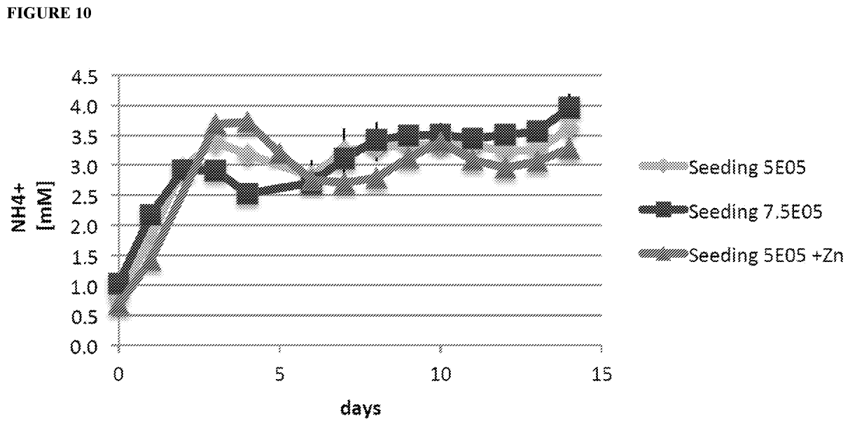

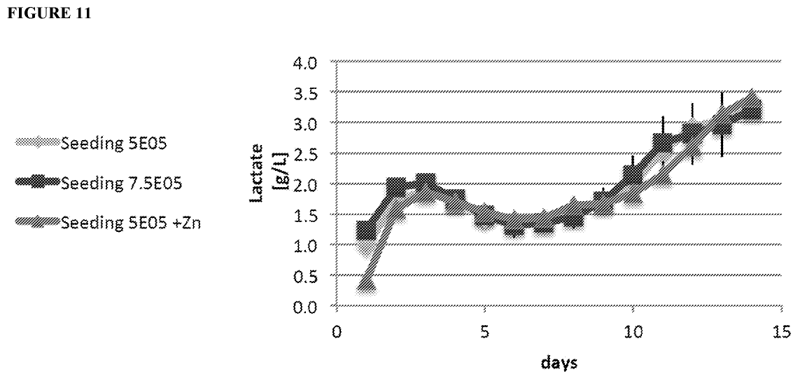

pH was measured over the course of the bioreactor process for IAP produced under all three conditions, and the results are shown in FIG. 7. Osmolality was also measured over the course of the bioreactor process for IAP produced under all three conditions, and the results are shown in FIG. 8. Daily measurements of insulin, ammonium, and lactate levels for IAP produced under all three conditions are depicted in FIG. 9, FIG. 10, and FIG. 11, respectively.

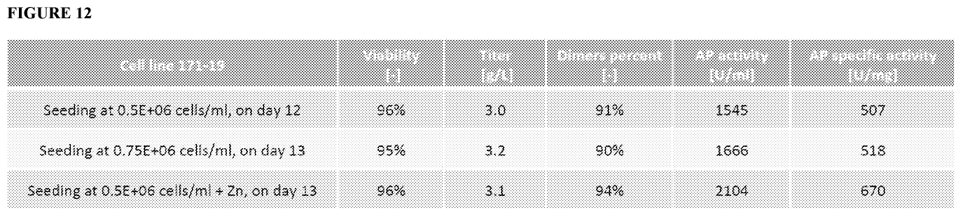

Condition 3′s surprising results are further depicted in FIG. 12, in which cell viability, titer (g/L), percent dimers, AP activity (U/mL), and AP specific activity (U/mg) are shown as of day 12 for Condition 1 and as of day 13 for Conditions 2 and 3. Both AP activity and AP specific activity were higher in results depicted under Condition 3 as opposed to Conditions 1 and 2.

### Example 3: Downstream Analysis of Large-Scale Bioreactor IAP Production

IAP was produced in large-scale bioreactors at 3 L, 50 L, and 200 L. Product quality measurements were conducted on the IAP product for each of the various runs.

For each bioreactor size, the runs included an initial amount of ZnSO₄ and MgCl₂ added per liter of medium. For the 3 L reactor, 40 μM solution ZnSO₄ was initially added per liter of medium, and 1 mL of 1M MgCl₂ was initially added per Kg of medium. For the 50 L reactor, 40 μL of 2M solution ZnSO₄ was initially added per liter of medium, and 1 mL of 1M MgCl₂ was initially added per Kg of medium. For the 200 L reactor, 40 μL of 2M solution ZnSO₄ was initially added per liter of medium, and 1 mL of 1M MgCl₂ was initially added per Kg of medium.

On Day 11 of each run, an addition of 40mM solution of ZnSO₄ was conducted in order to bring the concentrations to established levels: the 3 L reactor concentration brought to 80 μM; the 50 L reactor concentration brought to 100 μM; and the 200 L reactor concentration brought to 100 μM. No additions of MgCl₂ were made during each of the runs.

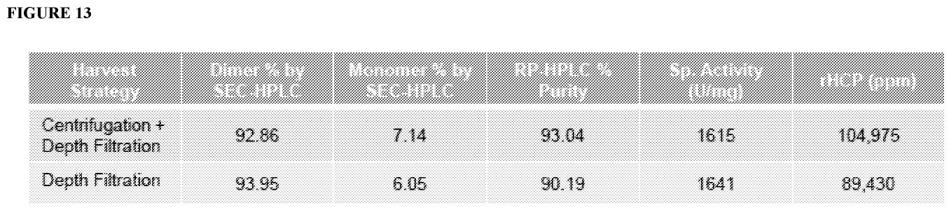

Product quality measurements (e.g., dimer % by SEC-HPLC, monomer % by SEC-HPLC, RP-HPLC% purity, specific activity, and rHCP) were analyzed for the 3 L bioreactor run, and the results are depicted in FIG. 13.

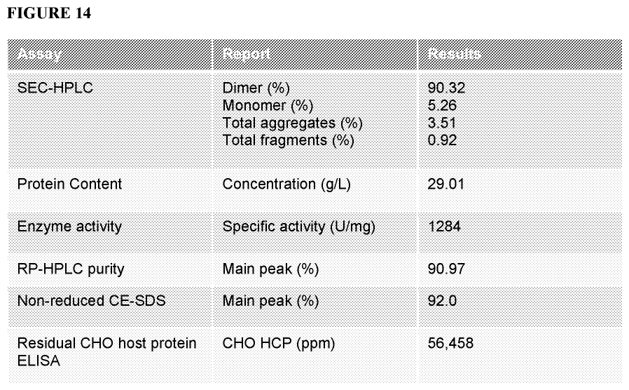

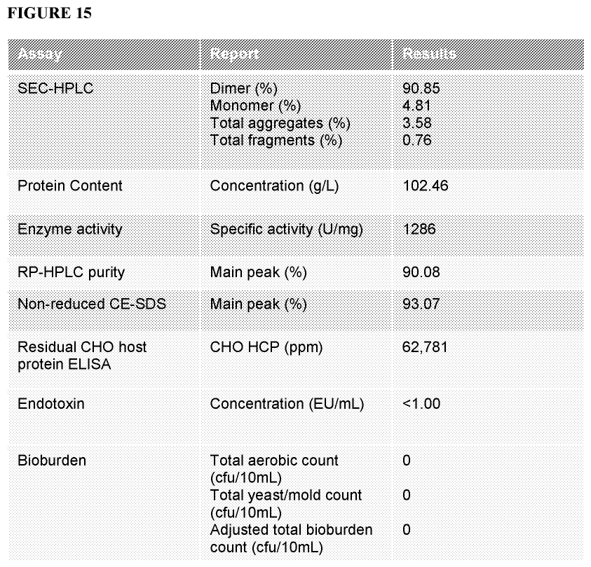

FIG. 14 and FIG. 15 depict product quality measurements for the 50 L and 200 L bioreactor runs, respectively. Assays included SEC-HPLC, protein content, enzyme activity, RP-HPLC activity, non-reduced CE-SDS, and residual host cell protein ELISA.

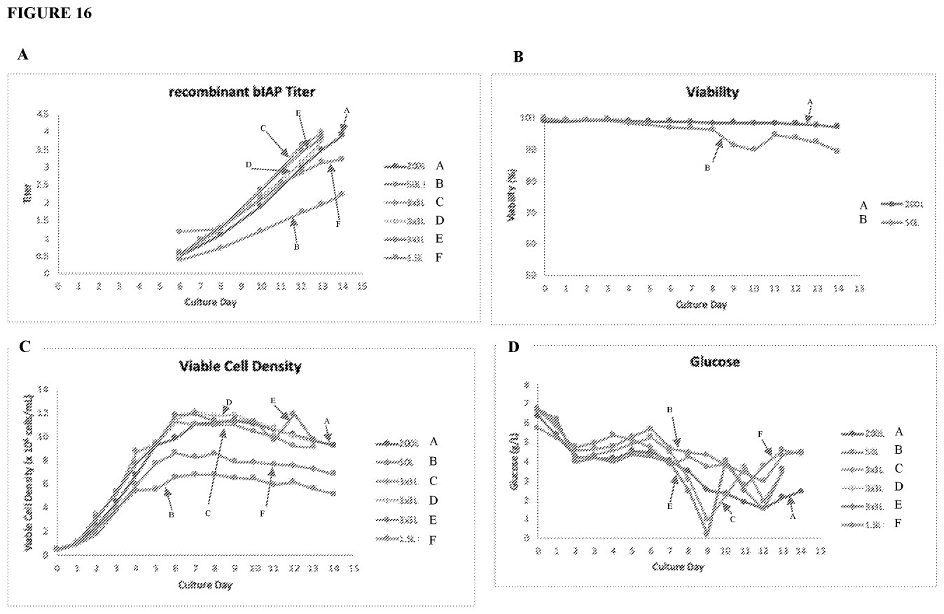

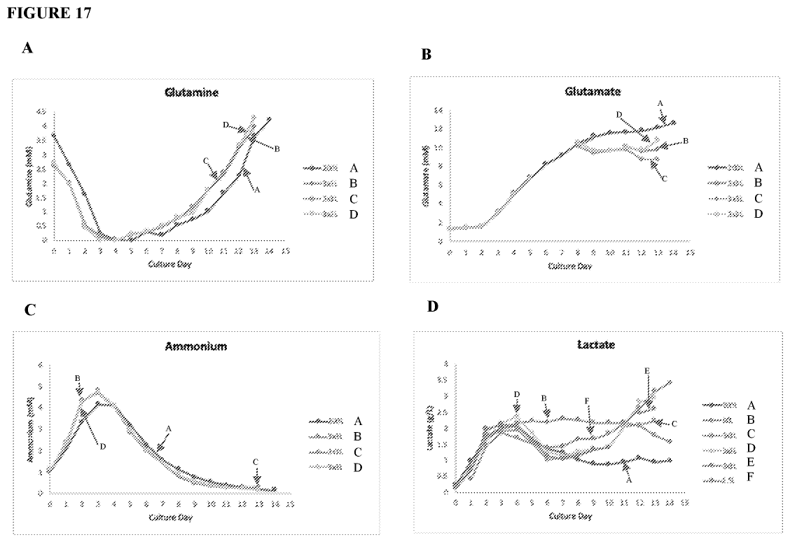

An analysis of various cell culture parameters (such as recombinant bIAP titer, viability, viable cell count density, and glucose, glutamine, glutamate, ammonium, and lactate amounts) was also conducted, with the results shown in FIGS. 16A-D and 17A-D.

### DEFINITIONS

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents (e.g., beta-lactamases and/or additional therapeutic agents described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

The term “bioreactor,” as used herein, refers to a vessel used for the growth of a host cell culture. A bioreactor can be of any size so long as it is useful for the culturing of mammalian cells. Typically, a bioreactor will be at least 1 liter and may be 10, 100, 250, 500, 1000, 2500, 5000, 8000, 10,000, 12,0000 liters or more, or any volume in between. Internal conditions of a bioreactor, including, but not limited to pH, osmolarity, CO₂ saturation, O₂ saturation, temperature and combinations thereof, are typically controlled during the culturing period. A bioreactor can be composed of any material that suitable for holding cells in media under the culture conditions of the present invention, including glass, plastic or metal. In some embodiments, a bioreactor may be used for performing animal cell culture. In some embodiments, a bioreactor may be used for performing mammalian cell culture. In some embodiments, a bioreactor may be used with cells and/or cell lines derived from such organisms as, but not limited to, mammalian cell, insect cells, bacterial cells, yeast cells and human cells. In some embodiments, a bioreactor is used for large-scale cell culture production and is typically at least 100 liters and may be 200, 500, 1000, 2500, 5000, 8000, 10,000, 12,0000 liters or more, or any volume in between. One of ordinary skill in the art will be aware of and will be able to choose suitable bioreactors for use in practicing the present invention.

The term “cell density” as used herein, refers to that number of cells present in a given volume of medium.

As used herein, “fed-batch culture” refers to a method of culturing cells in which additional components are provided to the culture at some time subsequent to the beginning of the culture process. As used herein, these additional components provided to the culture at some time subsequent to the beginning of the culturing process are referred to as “feed”s. The provided components typically comprise nutritional supplements for the cells which have been depleted during the culturing process. A feed can also be a chemically-defined formula. A fed-batch culture is typically stopped at some point and the cells and/or components in the medium are harvested and/or separated and optionally purified.

The term “integrated viable cell density (IVCD),” as used herein, refers to the average density of viable cells over the course of the culture multiplied by the amount of time the culture has run. Assuming the amount of polypeptide and/or protein produced is proportional to the number of viable cells present over the course of the culture, integrated viable cell density is a useful tool for estimating the amount of polypeptide and/or protein produced over the course of the culture.

The term “medium” as used herein refers to a solution containing nutrients which nourish growing cells. Typically, these solutions provide essential and non-essential amino acids, vitamins, energy sources, lipids, and trace elements required by the cell for minimal growth and/or survival. The solution may also contain components that enhance growth and/or survival above the minimal rate, including hormones and growth factors. In some embodiments, medium is formulated to a pH and salt concentration optimal for cell survival and proliferation. In some embodiments, medium may be a “chemically defined medium”—a serum-free media that contains no proteins, hydrolysates or components of unknown composition. In some embodiment, chemically defined medium is free of animal-derived components and all components within the medium have a known chemical structure. In some embodiments, medium may be a “serum based medium,” e.g. a medium that has been supplemented with animal derived components such as, but not limited to, fetal calf serum, horse serum, goat serum, donkey serum and/or combinations thereof.

The term “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

“Osmolality” is a measure of the osmotic pressure of dissolved solute particles in an aqueous solution. The solute particles include both ions and non-ionized molecules. Osmolality is expressed as the concentration of osmotically active particles (i.e., osmoles) dissolved in 1 kg of solution. For example, 1 mOsm/kg H₂O at 38° C. is equivalent to an osmotic pressure of 19 mm Hg. “Osmolarity,” by contrast, refers to the number of solute particles dissolved in 1 liter of solution. When used herein, the abbreviation “mOsm” means “milliosmoles/kg solution.”

The term “seeding” as used herein refers to the process of providing a cell culture to a bioreactor or another vessel for cell culture production. In some embodiments a “seed culture” is used, in which the cells have been propagated in a smaller cell culture vessel, i.e. Tissue-culture flask, Tissue-culture plate, Tissue-culture roller bottle, etc., prior to seeding. Alternatively, in some embodiments, the cells may have been frozen and thawed immediately prior to providing them to the bioreactor or vessel. The term refers to any number of cells, including a single cell.

The term “titer” as used herein refers to the total amount of expressed polypeptide or protein produced by a cell culture divided by a given amount of medium volume.

As used herein, the term “viable cell density (VCD)” refers to the number of living cells per unit volume.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of producing a recombinant IAP comprising:
a) providing an initial culture of mammalian cells, the mammalian cells comprising a gene encoding said IAP;
b) batch feeding said culture;
c) adding a quantity of supplemental zinc to said culture; and
d) isolating the produced IAP from said mammalian cells.

2. The method of claim 1, wherein said supplemental zinc is selected from the group consisting of ZnSO₄, ZnCl₂, ZnBr₂, zinc citrate, hydrolysate, and plasma zinc bound to serum albumin.

3. The method of claim 2, wherein said batch feeding occurs during the culturing process.

4. The method of claim 3, wherein said batch feeding occurs at least once every day during the culturing process.

5. The method of any one of the preceding claims, wherein said batch feeding is terminated at least 10 days after initiation of the culturing process.

6. The method of any one of the preceding claims, wherein said batch feeding comprises at least two separate feeds.

7. The method of claim 6, wherein the ratio of feeding of the first feed to the second feed is about 10:1.

8. The method of any one of the preceding claims, wherein the addition of supplemental zinc occurs at least once during the culturing process.

9. The method of any one of the preceding claims, wherein the addition of supplemental zinc occurs about 11 days after initiation of the culturing process.

10. The method of any one of the preceding claims, wherein said quantity of supplemental zinc is between about 60 to 100 μM zinc.

11. The method of claim 2, wherein said quantity of supplemental zinc is between about 60 to 100 μM zinc.

11. The method of any one of the preceding claims, wherein said quantity of supplemental zinc is about 80 μM zinc.

12. The method of any one of the preceding claims, wherein said cells are separated from the produced IAP by at least 13 days after the initiation of the culturing process.

13. The method of any one of the preceding claims, wherein the method occurs in a bioreactor.

14. The method of any one of the preceding claims, wherein said mammalian cells are CHO cells.

15. The method of any one of the preceding claims, wherein said initial culture comprises glutamine, sodium hypoxanthine and thymidine (HT), ZnSO₄, MgCl₂, a poloxamer, and antifoam.

16. The method of any one of the preceding claims, wherein the provided culture comprises a seeding density of at least 0.5×10⁶ cells/mL.

17. The method of any one of the preceding claims, wherein at least one temperature shift occurs during the culturing process.

18. The method of any one of the preceding claims, wherein a first temperature shift occurs at about 72 hours after initiation of the culturing process.

19. The method of any one of the preceding claims, wherein the first temperature shift comprises a temperature decrease from about 37° C. to about 33° C.

20. The method of any one of the preceding claims, wherein a second temperature shift occurs at about 288 hours after initiation of the culturing process.

21. The method of any one of the preceding claims, wherein the second temperature shift comprises a temperature decrease from about 33° C. to about 31° C.

22. The method of any one of the preceding claims, wherein the IAP is bovine IAP.

23. The method of claim 22, wherein the bovine IAP comprises an amino acid sequence having at least 90% identity with any one of SEQ ID NO: 2, SEQ ID NO: 3, SEQ ID NO: 5, SEQ ID NO: 6, and SEQ ID NO: 7.

24. The method of claim 23, wherein the bovine IAP comprises an amino acid sequence having at least 95% identity with SEQ ID NO: 2.

25. The method of claim 24, wherein the bovine IAP comprises the amino acid sequence of SEQ ID NO: 2.

26. The method of claim 23, wherein the bovine IAP comprises an amino acid sequence having at least 95% identity with SEQ ID NO: 3.

27. The method of claim 26, wherein the bovine IAP comprises the amino acid sequence of SEQ ID NO: 3.

28. The method of any one of the preceding claims, wherein said recovered IAP has a specific activity of at least 600 U/mg.

29. The method of claim 11, wherein said recovered IAP has a specific activity of at least 600 U/mg.

30. The method of any one of the preceding claims, wherein said recovered IAP has a specific activity of about 618 U/mg.

31. The method of any one of the preceding claims, wherein said recovered IAP has a total AP activity of at least 1,980 U/mL.

32. The method of any one of the preceding claims, wherein said recovered IAP has a total AP activity of about 1,994 U/mL.

33. The method of any one of the preceding claims, wherein said recovered IAP comprises about 2.75×10⁶ total active AP units.

34. A method of producing a recombinant IAP comprising:
a) providing an initial culture of mammalian cells comprising a gene encoding said IAP;
b) batch feeding said culture over 12 days, wherein said feeding initiates at around day 2 and is terminated at around day 13;
c) adding a quantity of supplemental zinc to said culture at around day 11 after initiation of the culturing process; and
d) isolating the produced IAP from said mammalian cells by at least day 13 after initiation of the culturing process.

35. The method of claim 34, wherein said supplemental zinc is selected from the group consisting of ZnSO₄, ZnCl₂, ZnBr₂, zinc citrate, hydrolysate, and plasma zinc bound to serum albumin.

36. The method of either one of claim 34 or 35, wherein said batch feeding occurs at least once every day.

37. The method of any one of claims 34-36, wherein said batch feeding comprises at least two separate feeds.

38. The method of any one of claims 34-37, wherein the ratio of feeding of the first feed to the second feed is about 10:1.

39. The method of any one of claims 34-38, wherein said quantity of supplemental zinc is between about 60 to 100 μM ZnSO₄.

40. The method of claim 34, wherein said quantity of supplemental zinc is between about 60 to 100 μM ZnSO₄.

41. The method of any one of claims 34-40, wherein said quantity of supplemental zinc is about 80 μM ZnSO₄.

42. The method of any one of claims 34-41, wherein the process occurs in a bioreactor.

43. The method of any one of claims 34-42, wherein said mammalian cells are CHO cells.

44. The method of any one of claims 34-43, wherein said initial culture comprises glutamine, sodium hypoxanthine and thymidine (HT), ZnSO₄, MgCl₂, a poloxamer, and antifoam.

45. The method of any one of claims 34-44, wherein the provided culture comprises a seeding density of at least 0.5×10⁶ cells/mL.

46. The method of any one of claims 34-45, wherein at least one temperature shift occurs during the culturing process.

47. The method of any one of claims 34-46, wherein a first temperature shift occurs at about 72 hours after initiation of the culturing process.

48. The method of any one of claims 34-47, wherein the first temperature shift comprises a temperature decrease from about 37° C. to about 33° C.

49. The method of any one of claims 34-48, wherein a second temperature shift occurs at about 288 hours after the initial culture is provided.

50. The method of any one of claims 34-49, wherein the second temperature shift comprises a temperature decrease from about 33° C. to about 31° C.

51. The method of any one of claims 34-50, wherein the IAP is bovine IAP.

52. The method of claim 51, wherein the bovine IAP comprises an amino acid sequence having at least 90% identity with any one of SEQ ID NO: 2, SEQ ID NO: 3, SEQ ID NO: 5, SEQ ID NO: 6, and SEQ ID NO: 7.

53. The method of claim 52, wherein the bovine IAP comprises an amino acid sequence having at least 95% identity with SEQ ID NO: 2.

54. The method of claim 53, wherein the bovine IAP comprises the amino acid sequence of SEQ ID NO: 2.

55. The method of claim 52, wherein the bovine IAP comprises an amino acid sequence having at least 95% identity with SEQ ID NO: 3.

56. The method of claim 55, wherein the bovine IAP comprises the amino acid sequence of SEQ ID NO: 3.

57. The method of claim 34, wherein the IAP is bovine IAP.

58. The method of any one of claims 34-57, wherein said recovered IAP has a specific activity of at least 600 U/mg.

59. The method of claim 34, wherein said recovered IAP has a specific activity of at least 600 U/mg.

60. The method of any one of claims 34-59, wherein said recovered IAP has a specific activity of about 618 U/mg.

61. The method of any one of claims 34-60, wherein said recovered IAP has a total AP activity of at least 1,980 U/mL.

62. The method of any one of claims 34-61, wherein said recovered IAP has a total AP activity of about 1,994 U/mL.

63. The method of any one of claims 34-62, wherein said recovered IAP comprises about 2.75×10⁶ total active AP units.

64. The method of any one of the preceding claims, wherein 1mM of MgCl₂ is added to the initial culture medium.

65. The methof of any one of the preceding claims, wherein the culturing process occurs in a bioreactor.

66. The method of claim 65, wherein the bioreactor comprises a size of 3 L, 50 L, or 200 L.

67. The method of any one of the preceding claims, wherein the IAP comprises at least 90% purity as measured by RP-HPLC.

68. A method of producing a recombinant IAP comprising:
a) providing an initial culture of mammalian cells comprising a gene encoding said IAP at day 1;
b) seeding said culture of mammalian cells in a bioreactor at a seeding density of at least 0.5×10⁶ cells/mL;
c) batch feeding said culture, wherein said feeding initiates at about day 2;
d) adjusting the temperature a first time from about 37° C. to about 33° C. on about day 3;
e) adjusting the pH of said culture to about 6.85 on day 3;
f) batch feeding said culture at least once every day from about day 2 to about day 13;
g) adding a quantity of about 80 μM ZnSO₄ supplemental zinc to said culture at about day 11;
h) adjusting the temperature a second time from about 33° C. to about 31° C. on about day 12; and
i) isolating the produced IAP from said mammalian cells by at least day 13.
